(19) United States
(12) Patent Application Publication
(10) Pub. No.: US 2024/0026166 A1
(43) Pub. Date: Jan. 25, 2024

(54) COATING OF MATERIALS WITH BIOSURFACTANT COMPOUNDS

(71) Applicant: Stellenbosch University, Stellenbosch, Western Cape Province (ZA)

(72) Inventors: Wesaal Khan, Stellenbosch, Western Cape Province (ZA); Thando Ndlovu, Stellenbosch, Western Cape Province (ZA); Tanya Lee Clements, Stellenbosch, Western Cape Province (ZA); Nusrat Begum Mutta, Stellenbosch, Western Cape Province (ZA)

(21) Appl. No.: 18/028,396

(22) PCT Filed: Sep. 17, 2021
(86) PCT No.: PCT/ZA2021/050053

§ 371 Date: Mar. 24, 2023

(30) Foreign Application Priority Data
Sep. 25, 2020 (ZA) 2020/05912

(51) Int. Cl. C09D 5/16; A01N 25/10; A01N 63/22; A01N 63/27; A01N 25/24; A01P 1/00; A01N 25/30

(52) U.S. Cl. CPC C09D 5/1625; A01N 25/10; A01N 63/22; A01N 63/27; A01N 25/24; A01P 1/00; A01N 25/30

(57) ABSTRACT

The invention provides a coating method for coating a surface of a substrate material with a biosurfactant. The method includes the following steps: modifying the biosurfactant to promote its reactivity with a silane linker; oxidising the surface of the substrate material; functionalising the surface of the substrate material with a silane linker; and reacting the modified biosurfactant with the functionalised surface. The biosurfactant becomes covalently bonded to the surface of the substrate material. The substrate material may be a polymer such as high-density polyethylene or polyvinyl chloride, or a ferrous metal such as stainless steel. The biosurfactant may be produced by one or more bacterial strains selected from the group consisting of Pseudomonas aeruginosa, Bacillus amyloliquefaciens and Serratia marcescens. The invention also provides articles of manufacture which include substrate materials that are at least partially coated with biosurfactants. The substrate materials and biosurfactants may be as described above.

### FIELD OF THE INVENTION

This invention relates to antimicrobial biosurfactant compounds and their use as surface coatings with antifouling activity. It relates in particular, but not exclusively, to methods and protocols for coating polymeric and metallic materials with biosurfactant compounds to inhibit biofilm formation and reduce biofouling. The invention extends to materials and articles of manufacture coated with biosurfactant compounds.

### BACKGROUND TO THE INVENTION

Biosurfactant compounds (“biosurfactants”) are surface-active amphiphilic molecules that preferentially partition at the interface between different phases, i.e. in different fluid phases such as oil/water or air/water interfaces. They are non-ribosomally synthesised by certain bacteria, fungi and yeast during secondary metabolism. They can alter surface properties such as charge and hydrophobicity, thereby interfering with bacterial-surface and/or bacterial-bacterial interactions. These properties are advantageous as they can be exploited as biofilm disrupting and antiadhesive agents, amongst others. Classes of biosurfactants include glycolipids, lipopeptides, lipoproteins, phospholipids, polymeric surfactants and particulate surfactants.

Strategies to inhibit the formation of biofilms on surfaces include the use of antifouling agents such as biosurfactants, polyethylene glycol (PEG), zwitterionic polymers, and topographic surfaces. One of the applications of biosurfactants is therefore to regulate the attachment to, and removal of, microorganisms from surfaces.

Biosurfactants have several advantages over their chemically synthesized counterparts. These may relate to their biodegradability, biocompatibility, low toxicity, digestibility, specificity, surface activity, tolerance to pH, temperature and ionic strength, emulsifying and demulsifying ability, foaming capacity and antimicrobial activity. They are typically more environmentally friendly than their synthetic counterparts and can exhibit a higher efficiency at lower concentrations. Furthermore, feedstocks for producing biosurfactants are readily available and renewable. Biosurfactants may be more effective at inhibiting or disrupting biofilms than traditional inhibitory agents.

Glycolipids and lipopeptides are two classes of biosurfactants that can inhibit microbial adhesion to surfaces and disrupt preformed biofilms. Representative biosurfactants with good antiadhesive activity include rhamnolipids (RL) (a class of glycolipid produced inter alia by Pseudomonas aeruginosa) and surfactin (a lipopeptide produced inter alia by Bacillus subtilis). Rhamnolipids can disrupt preformed Bacillus pumilus biofilms on polystyrene microplates and are also effective against biofilms of Bordetella bronchiseptica. Surfactin can decrease the biofilm formation of Salmonella typhimurium, Salmonella enterica, Escherichia coli and Proteus mirabilis on polyvinyl chloride (PVC) plates and vinyl urethral catheters.

Biosurfactants produced by strains of Pseudomonas aeruginosa, Bacillus amyloliquefaciens and Serratia marcescens have antimicrobial activity and are capable of inhibiting biofilms. For example, congeners or homologues of rhamnolipids produced by a P. aeruginosa ST5 strain (e.g., di- and monorhamnolipid congeners such as RhaRhaC₁₀C₁₀ and RhaC₁₀C₁₀, RhaRhaC₈C₁₀/RhaRhaC₁₀C₈ and RhaC₈C₁₀/RhaC₁₀C₈, as well as RhaRhaC₁₂C₁₀/RhaRhaC₁₀C₁₂ and RhaC₁₂C₁₀/RhaC₁₀C₁₂) may display antimicrobial activity against a broad spectrum of opportunistic and pathogenic microorganisms, including antibiotic resistant Staphylococcus aureus (S. aureus) and Escherichia coli strains and the pathogenic yeast Candida albicans. Congeners or homologues of rhamnolipids (such as RhaC₁₀C₁₀, RhaC₁₂C₁₀ or RhaC₁₀C₁₂, RhaRhaC₁₀C₈ or RhaRhaC₈C₁₀, RhaRhaC₁₀C₁₀ and RhaRhaC₁₂C₁₀ or RhaRhaC₁₀C₁₂) may be produced by a P. aeruginosa EBN-8 strain and can be coated onto silver (Ag) and iron oxide (Fe₃O₄) nanoparticles. Synergistic antibacterial and antiadhesive properties of such rhamnolipids may contribute to anti-biofilm activity not only during biofilm formation but also against pre-formed biofilms of P. aeruginosa and S. aureus strains.

Cyclic lipopeptides produced by B. subtilis, such as surfactin and its analogues, may inhibit biofilm formation on medical and industrial objects. Surfactin can be used as a direct coating (in some cases baked onto the target surface) or as a coating formed by mixing the surfactin with paint or molten plastic. PVC has been described as a material that could be coated with lipopeptides, but there is no detailed teaching as to how such coating might be accomplished.

Glycolipids from S. marcescens may be used for inhibiting or disrupting biofilms. For example, if coated onto the surfaces of wells of a microtiter plate by simple adsorption methods, they may inhibit biofilm formation on those surfaces.

Glass, silicon, polymethylsiloxane (PDMS) and titanium can be coated with antimicrobial compounds. However, the antimicrobial compounds used to coat such materials are typically synthetic instead of biologically-produced.

Some biosurfactant coating methods rely upon simple absorption of the biosurfactant by the surface. However, this may not be effective to provide stable and durable coatings of biosurfactants.

Catheter surfaces can be treated with 3-triethoxysilylpropan-1-amine (APTES) and coated with enzymes to inhibit or disrupt biofilms. For example, the enzyme, cellobiose dehydrogenase (CDH) may be coated onto a polydimethylsiloxane (PDMS) surface using a step-wise procedure which involves the following steps: (1) plasma treatment, (2) grafting of APTES, (3) grafting of glutaraldehyde, and (4) grafting of the CDH enzyme.

There is a need for alternative methods of coating materials to reduce biofouling by inhibiting the adhesion of microorganisms and the formation of biofilms. Such materials may be suitable for use in industries such as the water, food, medical, industrial cooling and marine industries, amongst others.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

According to a first aspect of the invention there is provided a method for coating a surface of a substrate material with a biosurfactant, the method comprising modifying the biosurfactant to promote its reactivity with a silane linker; oxidising the surface of the substrate material; functionalising the surface of the substrate material with a silane linker; and reacting the modified biosurfactant with the functionalised surface, thereby covalently to bond the biosurfactant to the surface of the substrate material.

The substrate material may be selected from the group consisting of polymers and ferrous metals. The material may be selected from the group consisting of high-density polyethylene (HDPE), polyvinyl chloride (PVC), and stainless steel. The material may be a material which, prior to coating, is susceptible to formation of biofilms on its surface. The material may have properties required for apparatus selected from the group consisting of water distribution apparatus; apparatus configured for contact with food; medical apparatus and devices; industrial cooling apparatus; and shipping and marine apparatus. The material may comprise a piping material.

The biosurfactant may be produced by at least one strain of a species of microorganism selected from the group consisting of Pseudomonas aeruginosa, Bacillus amyloliquefaciens and Serratia marcescens.

The step of oxidising the surface of the material may include hydroxylating the surface. The hydroxylation step may be carried out by treating the material with a piranha solution to provide hydroxyl (—OH) groups on the surface of the material. The method may include a step of etching the surface.

For cases where the biosurfactant includes at least one carboxylic group, the step of modifying the biosurfactant to promote its reactivity with the silane linker may include an esterification step. The esterification step may include a Steglich esterification reaction step. Thus, the step of modifying the biosurfactant may include a step of functionalising the carboxylic group or groups of the biosurfactant by generating activated ester in the presence of N-Hydroxysuccinimide (NHS) under an anhydrous mild Steglich esterification reaction. This method may be appropriate for modifying rhamnolipid, surfactin and bacillomycin biosurfactants, such as those produced by Pseudomonas aeruginosa and Bacillus amyloliquefaciens strains. These biosurfactants have carboxylic groups.

For cases where the biosurfactant includes at least one hydroxyl group (—OH), the step of modifying the biosurfactant to promote its reactivity with the silane linker may comprise a step of functionalising the hydroxyl group of the biosurfactant by replacing it with a chlorine group (—Cl). The step of modifying the biosurfactant to promote its reactivity with the silane linker may include solubilising the biosurfactant and treating it with thionyl chloride and pyridine.

Advantageously, the silane linker used to functionalise the surface of the material may be 3-triethoxysilylpropan-1-amine (APTES).

The reaction step may include immobilising the biosurfactant on the surface of the material. The reaction step may include bioconjugating the biosurfactant and the material.

The biosurfactant may comprise at least one compound selected from the group consisting of lipopeptides, glycolipids and glucosamine derivatives.

The lipopeptide may be selected from the group consisting of surfactin and its analogues, bacillomycin L and bacillomycin D and their homologues, and serrawettin W1 and its homologues.

The glycolipid compound may be selected from the group consisting of rhamnolipids and rhamnolipid congeners.

The method may be appropriate for modifying serrawettin and glucosamine-derived biosurfactants, such as those produced by Serratia marcescens strains. These biosurfactants have hydroxyl groups. Accordingly, the biosurfactant may comprise a serrawettin W1 homologue.

The biosurfactant may comprise a serratamolide compound. The biosurfactant may comprise a glucosamine derivative.

The biosurfactant may have broad-spectrum antimicrobial activity. The biosurfactant may exhibit biofilm-inhibiting activity. The biosurfactant may have biofilm-inhibiting activity against Escherichia coli, Listeria monocytogenes and Cryptococcus neoformans (as when the biosurfactant is produced by either or both of Pseudomonas aeruginosa and Bacillus amyloliquefaciens). The biosurfactant may have biofilm-inhibiting activity against Pseudomonas aeruginosa and Enterococcus faecalis (as when the biosurfactant is produced by Serratia marcescens).

The biosurfactant may comprise a crude extract of biosurfactant compounds produced by at least one strain selected from the group consisting of Pseudomonas aeruginosa, Bacillus amyloliquefaciens and Serratia marcescens.

The biosurfactant may comprise a biosurfactant compound isolated from a biosurfactant crude extract produced by at least one strain selected from the group consisting of Pseudomonas aeruginosa, Bacillus amyloliquefaciens and Serratia marcescens. The biosurfactant may comprise a constituent of a purified fraction of the crude extract.

The coating method may include performing an extraction step to harvest a crude extract of biosurfactant compounds produced by at least one strain selected from the group consisting of Pseudomonas aeruginosa, Bacillus amyloliquefaciens and Serratia marcescens. The extraction step may include growing bacterial cells of the strain in a culture medium. It may include removing a bulk of the bacterial cells from the culture medium, thereby to yield a supernatant substantially free of the bacterial cells. The step of removing the bacterial cells may be conducted by centrifugation.

The extraction step may further include acidifying the supernatant, thereby to yield the crude extract of the biosurfactant compounds as a precipitate. It may include harvesting the precipitate by centrifugation.

The extraction step may include freeze drying the crude extract. Depending on whether or not the acid precipitation step has been performed, the freeze drying step may involve freeze drying the precipitate or the cell-free supernatant, respectively.

The extraction step may include at least partially purifying the crude extract, i.e. the precipitate or supernatant, as applicable, thereby to yield a purified crude extract of the biosurfactant compounds. The purification step may be performed by solvent extraction. The solvent employed for the solvent extraction may be selected from the group consisting of acetonitrile, chloroform-methanol, acetone, n-heptane, petroleum ether, ethyl acetate, n-hexane, ether, and n-octane. The solvent may advantageously comprise acetonitrile.

The method may further include fractionating the mixture of biosurfactant compounds to obtain fractions thereof. Each fraction may contain a different constituent biosurfactant compound of the mixture. The fractionation may be carried out by subjecting the mixture to high performance liquid chromatography.

According to a further aspect of the invention there is provided a protocol for immobilising a biosurfactant on a surface of a substrate material, the protocol including steps of modifying the biosurfactant to promote its reactivity with a silane linker; functionalising the surface of the substrate material with a silane linker; and reacting the modified biosurfactant with the functionalised surface, thereby covalently to bond the biosurfactant to the surface of the substrate material. The substrate material may be selected from the group consisting of polymers and ferrous metals. The biosurfactant may be produced by a strain of a microorganism selected from the group consisting of Pseudomonas aeruginosa, Bacillus amyloliquefaciens and Serratia marcescens.

The protocol may include a step of oxidising the surface of the substrate material prior to functionalisation. The step of oxidising the surface may comprise hydroxylating the surface. The protocol may include etching the surface.

Further details of the material and the biosurfactant may be as hereinbefore described. The silane linker may be APTES.

The steps of modifying the biosurfactant and of functionalising the surface of the material may be as hereinbefore described.

According to a further aspect of the invention there is provided a method of antifouling a surface of a material, which includes performing the steps of either or both of the coating method and immobilisation protocol as hereinbefore described.

The invention extends to a use of the described coating method for antifouling a surface of a material.

According to a further aspect of the invention there is provided a method of inhibiting formation of a biofilm on a surface of a material, which includes performing the steps of either or both of the coating method and immobilisation protocol as hereinbefore described.

The invention extends to a use of the described coating method for inhibiting formation of a biofilm on the surface of a material.

According to a further aspect of the invention there is provided an article of manufacture comprising a substrate material having a biosurfactant covalently bonded to at least a portion of its surface by means of the coating method disclosed herein. Further details of the substrate material and the biosurfactant may be as hereinbefore described.

The invention extends, further, to an article of manufacture comprising a substrate material at least partially coated with an antiadhesive substance which comprises a serrawettin W1 homologue. The serrawettin W1 homologue may comprise a serratamolide compound. The substrate material may be as hereinbefore described.

The invention extends to an article of manufacture comprising a substrate material at least partially coated with an antiadhesive substance which comprises a glucosamine derivative. The substrate material may be as hereinbefore described.

The invention extends to an article of manufacture comprising a biosurfactant covalently bonded to a substrate material, wherein the substrate material is selected from the group consisting of polymers and ferrous metals. The biosurfactant may be as hereinbefore described.

The article of manufacture may be selected from the group consisting of water distribution apparatus, apparatus configured for contact with food, medical apparatus and devices, industrial cooling apparatus, shipping and marine apparatus, and raw materials for manufacturing said types of apparatus. The article of manufacture may comprise a pipe.

The article of manufacture may have antifouling activity. The article of manufacture may have antiadhesive activity.

According to a further aspect of the invention there is provided an article of manufacture comprising a substrate material at least partially coated with a biosurfactant; wherein the substrate material is selected from the group consisting of high-density polyethylene (HDPE), polyvinyl chloride (PVC) and stainless steel; and the biosurfactant is produced by at least one strain selected from the group consisting of Pseudomonas aeruginosa, Bacillus amyloliquefaciens and Serratia marcescens.

Further details of the substrate material, the biosurfactant and the article of manufacture may be as hereinbefore described. The article of manufacture may have antifouling activity. The article of manufacture may have antiadhesive activity.

Modes of performing the invention will now be described, by way of example only, with reference to the accompanying figures.

### BRIEF DESCRIPTION OF THE FIGURES

In the figures:

FIG. 1 is a series of bar charts illustrating a reduction in adhesion of Escherichia coli (E. coli) to surfaces of a Minimum Biofilm Eradication Concentration (MBEC™) assay precoated with biosurfactant crude extracts from Pseudomonas aeruginosa and Bacillus amyloliquefaciens strains at various concentrations;

FIG. 2 is a series of bar charts illustrating a reduction in adhesion of Listeria monocytogenes (L. monocytogenes) to surfaces of an MBEC™ assay precoated with biosurfactant crude extracts from Pseudomonas aeruginosa and Bacillus amyloliquefaciens strains at various concentrations;

FIG. 3 is a series of bar charts illustrating a reduction in adhesion of a Cryptococcus neoformans (C. neoformans) strain to surfaces of an MBEC™ assay precoated with biosurfactant crude extracts from Pseudomonas aeruginosa and Bacillus amyloliquefaciens strains at various concentrations;

FIG. 4 is a series of bar charts illustrating a reduction in adhesion of a Pseudomonas aeruginosa strain to surfaces of an MBEC™ assay precoated with biosurfactant crude extracts from Serratia marcescens strains at various concentrations;

FIG. 5 is a series of bar charts illustrating a reduction in adhesion of an Enterococcus faecalis (E. faecalis) strain to surfaces of an MBEC™ assay precoated with biosurfactant crude extracts from Serratia marcescens strains at various concentrations;

FIG. 6 is a series of bar charts illustrating antifouling activity based on colony forming units and gene copies/mL of L. monocytogenes attached to uncoated materials and to materials coated with biosurfactant crude extracts from Pseudomonas aeruginosa and Bacillus amyloliquefaciens strains, analysed using plate counts (culture-based analysis) and EMA-qPCR analysis;

FIG. 7 is a series of bar charts illustrating antifouling activity based on colony forming units and gene copies/mL of E. faecalis attached to uncoated materials and to materials coated with biosurfactant crude extracts from Serratia marcescens strains, analysed using plate counts (culture-based analysis) and EMA-qPCR analysis; and

FIG. 8 is a series of bar charts illustrating antifouling activity based on colony forming units and gene copies/mL of P. aeruginosa attached to uncoated materials and to materials coated with biosurfactant crude extracts from Serratia marcescens strains, analysed using plate counts (culture-based analysis) and EMA-qPCR analysis.

### DETAILED DESCRIPTION WITH REFERENCE TO THE FIGURES

Biosurfactants produced by Pseudomonas aeruginosa, Bacillus amyloliquefaciens and Serratia marcescens strains display antimicrobial activity against a broad range of microorganisms. Analysis using the Minimum Biofilm Eradication Concentration (MBEC™) assay also indicates that they display biofilm disrupting and antiadhesive properties.

The method and protocol described herein may permit the coating of materials with these biosurfactants and others. The resultant coated materials may have antifouling properties.

Throughout this specification unless the context requires otherwise the terms ‘biosurfactant compound’ and ‘biosurfactant’ will be understood to mean a surface-active amphiphilic compound that is non-ribosomally synthesised during secondary metabolism by at least one microorganism selected from the group consisting of bacteria, fungi and yeast. The terms will be understood, further, to extend to a composition (e.g., an extract) comprising at least one such compound. Without limitation thereto, the scope of the terms will be understood to encompass glycolipids, lipopeptides, glycolipopeptide, lipoproteins, phospholipids, polymeric surfactants and particulate surfactants.

Glycolipids are a class of biosurfactants composed of a hydrophilic moiety made up of mono-, di-, tri- or tetra-saccharide carbohydrates, particularly although not exclusively galactose or glucose. These are attached to different (chain length) hydrophobic moieties which form a lipid backbone. Similar compounds are also found in the form of diacylglycerol glycosides, glucosylceramides and sterylglycosides attached to various phospholipid bilayer backbones of molecules which occur in animals, bacteria, fungi and plants. The most common glycolipid-based biosurfactants include rhamnolipids, rubiwettins, mannosylerythritol lipids, sophorolipids and trehalolipids. Several congeners/homologues exist for each glycolipid variant due to the varying lengths and isomers of the fatty acid chain, which confer considerable structural heterogeneity. The varying number and type of carbohydrate residues found at specific locations within the hydrophilic moiety contribute to the existence of congeners of each glycolipid.

Lipopeptide biosurfactants are a diverse group of low molecular weight (500-2 000 Da) compounds composed of short linear or cyclic peptides linked to fatty acids of varying length. Lipopeptides are classified into different families, which include surfactin, fengycin, iturin, serrawettin, subtilin, arthrofactin, polymoxyins, lichenysin, gramicidin, viscosin, kurstatin and peptide-lipid like compounds. Several homologues exist for each lipopeptide variant due to the varying lengths and isomers of the fatty acid chain (hydrophobic moiety), which confer structural heterogeneity. The peptide section of the lipopeptides is composed of varying amino acid residues found at specific locations within the hydrophilic moiety, which contribute to the existence of analogues of each lipopeptide.

According to the method described herein, the biosurfactants may be chemically modified to promote their reactivity. The surface of the material to be coated may be oxidised and functionalised with a silane linker. Thereafter, the modified biosurfactant may be reacted with the functionalised surface of the material. This may promote covalent bonding of the biosurfactant to the surface of the material.

The step of oxidising the surface may include hydroxylating the surface. The hydroxylation may be carried out by treating the material with a piranha etch solution.

The step of functionalising the surface may be performed using APTES as the silane linker.

The material to be coated may advantageously be selected from the group consisting of high-density polyethylene (HDPE), polyvinyl chloride (PVC), and stainless steel. The material may be one that is susceptible to the formation of biofilms on its surface. It may be of a type used for apparatus in the following classes: apparatus configured for water conveying, distribution and storage; apparatus configured for contact with food; medical apparatus and devices; industrial cooling apparatus; and shipping and marine apparatus. The material may comprise a piping material, for example.

The biosurfactant may comprise at least one compound selected from the group consisting of lipopeptides, glycolipids, and glucosamine derivatives.

The biosurfactant may comprise a lipopeptide selected from the group consisting of surfactin and its analogues, bacillomycin L and bacillomycin D and their homologues, and serrawettin W1 and its homologues. The biosurfactant may comprise a glycolipid selected from the group consisting of rhamnolipids and rhamnolipid congeners.

The biosurfactant may have broad-spectrum antimicrobial activity. The biosurfactant may have antimicrobial activity against Escherichia coli, Listeria monocytogenes and Cryptococcus neoformans (as when the biosurfactant is produced by either or both of Pseudomonas aeruginosa and Bacillus amyloliquefaciens). The biosurfactant may have antimicrobial activity against Pseudomonas aeruginosa and Enterococcus faecalis (as when the biosurfactant is produced by Serratia marcescens).

The biosurfactant may comprise a biofilm-inhibiting compound.

The described coating method may include a protocol for immobilising a biosurfactant on a surface of a material selected from the group consisting of polymers and ferrous metals, the biosurfactant being advantageously produced by a species of microorganism selected from the group consisting of Pseudomonas aeruginosa, Bacillus amyloliquefaciens, and Serratia marcescens. The protocol may include steps of modifying the biosurfactant to promote its reactivity; functionalising the surface of the material with a silane linker (e.g., APTES); and reacting the modified biosurfactant with the functionalised surface, thereby covalently to bond the biosurfactant to the surface of the material.

The protocol may include a step of oxidising the surface of the material prior to functionalisation. This may include hydroxylating the surface with a piranha solution.

Oxidising methods involving piranha solution—and functionalising methods involving APTES—may previously have been disclosed for purposes of coating of materials with peptides and other compounds; however, biosurfactants have not been immobilised on surfaces using the described method and protocol. In particular, biosurfactant compounds from Pseudomonas aeruginosa, Bacillus amyloliquefaciens and Serratia marcescens strains have not previously been immobilised on surfaces using the described method and protocol. The chemical modification of the selected biosurfactant compounds prior to immobilisation has not previously been applied for immobilisation, for example.

In addition, serrawettin W1 homologues (serratamolides) and glucosamine derivatives have not previously been utilised for antiadhesive purposes or applied as coating agents on piping materials.

### Example

Biosurfactants produced by five strains of Pseudomonas aeruginosa, Bacillus amyloliquefaciens and Serratia marcescens were used in the following exemplary mode of performing the coating method and immobilisation protocol. It will be appreciated that the foregoing list of bacterial strains is not intended to be closed or limiting; it is presented for illustrative purposes only. Biosurfactants produced by other bacterial strains may also fall within the scope of the invention.

The five bacterial strains used to produce the biosurfactants in this example may be identified by the following acronyms:
SB24 means a strain of Pseudomonas aeruginosa (P. aeruginosa)
ST34 means a first strain of Bacillus amyloliquefaciens (B. amyloliquefaciens)
SB12 means a second strain of Bacillus amyloliquefaciens
P1 means a pigmented strain of Serratia marcescens (S. marcescens)
NP1 means a non-pigmented strain of Serratia marcescens

The origins of the abovementioned strains were as follows:
B. amyloliquefaciens ST34 and SB12 strains: Deposited in the South African Rhizobium Culture Collection (SARCC), which is in the Plant Health and Protection Research Institute at the Agricultural Research Council (ARC) in Pretoria in the Republic of South Africa. The ST34 and SB12 strains were respectively allocated the numbers SARCC-696 and SARCC-812 as code identifiers in the culture collection. The strains are accessible to researchers by contacting the curator of the South African Rhizobium Culture Collection at the ARC.
P. aeruginosa SB24 strain: Deposited in the South African Rhizobium Culture Collection (SARCC), which is in the Plant Health and Protection Research Institute at the Agricultural Research Council (ARC) in Pretoria in the Republic of South Africa. The SB24 strain was allocated the number SARCC-3058 as a code identifier in the culture collection. The strain is accessible to researchers by contacting the curator of the South African Rhizobium Culture Collection at the ARC.
S. marcescens P1 and NP1 strains: Deposited in the South African Rhizobium Culture Collection (SARCC), which is in the Plant Health and Protection Research Institute at the Agricultural Research Council (ARC) in Pretoria in the Republic of South Africa. The P1 and NP1 strains were respectively allocated the numbers SARCC-3059 and SARCC-3060 as code identifiers in the culture collection. The strains are accessible to researchers by contacting the curator of the South African Rhizobium Culture Collection at the ARC.

### 1. MATERIALS

All chemicals used were reagent grade and used without further purification. The reactions were carried out in dry ethanol, unless stated otherwise. N-hydroxysuccinimide (NHS), 3-butyn-2-ol, 4-(dimethylamino)pyridine (DMAP), N,N′dicyclohexylcarbodiimide (DCC), APTES (99%), anhydrous magnesium sulphate, thionyl dichloride (97%, SOCl₂), pyridine, hydrogen peroxide solution (H₂O₂, 30% v/v in water), and concentrated sulphuric acid (H₂SO₄, 95-97%) were used as received without further purification.

Biosurfactant crude extracts were obtained from P. aeruginosa SB24. The extracts comprised rhamnolipid homologues. Additional biosurfactant crude extracts were obtained from B. amyloliquefaciens ST34 and SB12. These extracts comprised surfactin and bacillomycin L or bacillomycin D, respectively. Additional biosurfactant crude extracts were also obtained from S. marcescens P1 and NP1. These extracts comprised serrawettin W1 homologues and glucosamine derivative homologues, as well as prodigiosin (for the P1 crude extract only).

For the production of the biosurfactant compounds, Pseudomonas aeruginosa SB24 and Bacillus amyloliquefaciens ST34 and SB12 strains were first grown in baffled flasks containing mineral salt medium (MSM) supplemented with glucose or fructose for 120 hrs at 30° C. on an orbital shaker (120 rpm). Following the growth of SB24, ST34 and SB12, the bacterial cells were removed by centrifugation from the culture media (MSM). Subsequently, the SB24, ST34 and SB12 cell free supernatants were acidified by the addition of hydrochloric acid to obtain a pH of approximately two (2). The acidification process allowed the precipitation of the biosurfactant compounds, which were then harvested by centrifugation.

In contrast, the Serratia marcescens P1 and NP1 strains were grown in baffled flasks containing peptone glycerol (PG) broth for 120 hrs at 30° C. on an orbital shaker (120 rpm). Thereafter, the bacterial cells were removed by centrifugation from the culture media (PG broth) to obtain the cell free supernatant.

The acid precipitated extracts (SB24, ST34 and SB12) and cell free supernatants (P1 and NP1) were then freeze dried. The freeze dried samples were purified using two different solvent extraction methods [i.e. 70% acetonitrile or a chloroform-methanol (2:1, v/v) mixture]. The use of acetonitrile and chloroform-methanol extraction methods for the purification and extraction of the biosurfactant compounds produced by the SB24, ST34 and SB12 bacterial strains led to similar compounds being detected by an Ultra Performance Liquid Chromatography linked to electrospray ionization mass spectrometry (UPLC-ESI-MS) method. For example, the SB24 strain produced several rhamnolipid congeners/homologues, which were detected in both of the acetonitrile and chloroform-methanol crude extracts, but they varied in their relative abundance. The SB12 and ST34 bacterial strains produced several surfactin and bacillomycin analogues, which were detected in both of the acetonitrile and chloroform-methanol extracts; however, the relative abundance of each analogue varied. Due to the cost-effectiveness of the acetonitrile solvent and the reduced time required to conduct the extraction of biosurfactant produced by SB24, ST34 and SB12 bacterial strains, the acetonitrile was thus the preferred extraction method. In addition, the acetonitrile solvent extraction method resulted in the extraction of a higher number of serratamolide homologues in the P1 and NP1 crude extracts compared to the chloroform-methanol solvent extraction and was thus also the preferred extraction method. The recovery and purification of biosurfactants from aqueous media can be performed using liquid membrane (pertraction) processes. Other solvents such as acetone, n-heptane, petroleum ether, ethyl acetate, n-hexane, ether and n-octane can be used for the purification of biosurfactants; however, the use of such solvents can be costly and additional time is required to perform the purification using these solvents.

The minimum inhibitory concentrations (MIC) of the crude biosurfactant extracts produced by the SB24, ST34, SB12, P1 and NP1 strains were determined against selected Gram-negative and Gram-positive bacterial strains as well as fungal pathogens. The biofilm disruption and antiadhesive activity of the crude biosurfactant extracts was then determined using MBEC™ assay against susceptible bacterial and fungal strains (selected based on the MIC results). This assay was quantified using standard plate counts and ethidium monoazide bromide quantitative polymerase chain reaction (EMA-qPCR) analysis.

Based on the MBEC™ assay results, a selection of biosurfactant crude extracts were immobilised onto the surfaces of samples of high-density polyethylene (HDPE) PE300, polyvinyl chloride (PVC) and stainless steel grade 304, by performing the coating method described herein. The samples each measured about 20 mm×10 mm×1.5 mm.

The coated materials (and uncoated controls) were then exposed to L. monocytogenes C1 (for the SB24, ST34 and SB12 crude extracts) and Enterococcus faecalis (E. faecalis) S1 or Pseudomonas aeruginosa S1 68 (for the P1 and NP1 crude extracts) to investigate their antifouling properties. This analysis was carried out using standard culture-based techniques, EMA-qPCR and confocal laser scanning microscopy (CLSM) in conjunction with LIVE/DEAD viability stains to visually confirm the reduction of microbial adhesion.

The origins of the aforementioned test organisms were as follows:
L. monocytogenes C1: Stored and accessible in the Water Resource Laboratory culture collection in the Department of Microbiology at Stellenbosch University in the Republic of South Africa. The strain is available by contacting the chair of the Water Resource Laboratory.
E. faecalis S1: Stored and accessible in the Water Resource Laboratory culture collection in the Department of Microbiology at Stellenbosch University in the Republic of South Africa. The strain is available by contacting the chair of the Water Resource Laboratory.
P. aeruginosa S1 68: Stored and accessible in the Water Resource Laboratory culture collection in the Department of Microbiology at Stellenbosch University in the Republic of South Africa. The strain is available by contacting the chair of the Water Resource Laboratory.

### 2. ANTIADHESIVE ACTIVITY OF BIOSURFACTANTS

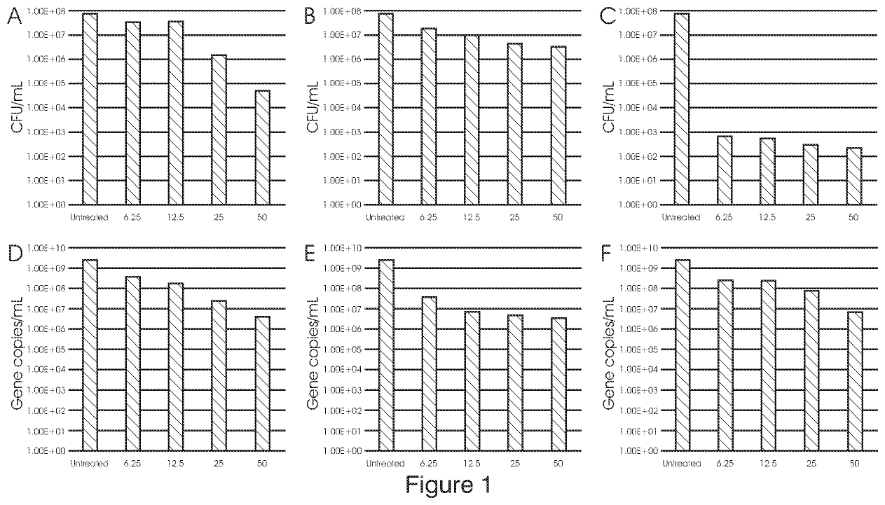

### 2.1. FIGS. 1-5: MBEC™ Assays

The first step in biofilm establishment is bacterial adhesion or adherence over the affected surface. Adhesion can be affected by various factors including type of microorganism, hydrophobicity and electrical charges of surface, environmental conditions, and the ability of microorganisms to produce extracellular polymers that help cells to anchor to surfaces.

Biosurfactants can alter the hydrophobicity of the surface which in turn affects the adhesion of microbes over the surface. The antiadhesive activity of the crude extracts (SB24, ST34, SB12, P1 and NP1) was evaluated utilising a Calgary Biofilm Device (CBD; MBEC™ Assay).

The results obtained for samples pre-treated with crude extracts produced by P. aeruginosa (SB24) and B. amyloliquefaciens (ST34 and SB12) strains will be discussed first for each test organism (E. coli L1, L. monocytogenes C1 and C. neoformans CAB 1055), followed by the results obtained for samples pre-treated with crude extracts obtained from S. marcescens (P1 and NP1) for each test organism (P. aeruginosa S1 68 and E. faecalis S1).

The origins of these test organisms were as follows:
E. coli L1: Stored and accessible in the Water Resource Laboratory culture collection in the Department of Microbiology at Stellenbosch University in the Republic of South Africa. The strain is available by contacting the chair of the Water Resource Laboratory.
L. monocytogenes C1: Stored and accessible in the Water Resource Laboratory culture collection in the Department of Microbiology at Stellenbosch University in the Republic of South Africa. The strain is available by contacting the chair of the Water Resource Laboratory.
Cryptococcus neoformans CAB 1055: Stored and accessible in the culture collection of the Department of Microbiology, University of Stellenbosch, Republic of South Africa. The strain is available by contacting the chair of the Water Resource Laboratory.
P. aeruginosa S1 68: Stored and accessible in the Water Resource Laboratory culture collection in the Department of Microbiology at Stellenbosch University in the Republic of South Africa. The strain is available by contacting the chair of the Water Resource Laboratory.
E. faecalis S1: Stored and accessible in the Water Resource Laboratory culture collection in the Department of Microbiology at Stellenbosch University in the Republic of South Africa. The strain is available by contacting the chair of the Water Resource Laboratory.
   2.1.1. Biosurfactant Crude Extracts Obtained from Pseudomonas and Bacillus Species (FIGS. 1-3)

Standard culture-based methods and EMA-qPCR analysis were used to evaluate the antiadhesive properties of the biosurfactant crude extracts produced by the P. aeruginosa (SB24) and B. amyloliquefaciens (ST34 and SB12) strains. The biosurfactant crude extracts were used for the pre-treatment of the surface of the pegs of the MBEC™ assay, at a concentration range of 6.25 to 50 mg/mL, to inhibit biofilm formation of E. coli L1, L. monocytogenes C1 and C. neoformans CAB 1055, respectively.

FIG. 1 illustrates a reduction in adhesion of E. coli L1 to surfaces of an MBEC™ assay precoated with biosurfactant crude extracts SB24, ST34 and SB12 at various concentrations (6.25, 12.5, 25 and 50 mg/mL). The E. coli cells capable of attaching to the coated surfaces were quantified using plate counts (culturing analysis) and EMA-qPCR analysis (an untreated control was included for each biosurfactant). The plate counts of E. coli L1 are presented in FIGS. 1A (SB24), 1B (ST34) and 1C (SB12). The EMA-qPCR analysis results of E. coli L1 are presented in FIGS. 1D (SB24), 1E (ST34), and 1F (SB12).

Referring to FIG. 1, the ability of the SB24 biosurfactant crude extract to inhibit adhesion of E. coli L1 is presented in FIG. 1A. For the untreated pegs, an average of 7.54×10⁷ CFU/mL E. coli L1 cells was enumerated in the biofilm suspension using culture-based analysis. For the pegs treated with the SB24 biosurfactant crude extracts at concentrations of 6.25, 12.5, 25 and 50 mg/mL, culture based analysis of the E. coli L1 cells in the biofilm suspension indicated that 3.67×10⁷ (99.51% inhibition), 3.80×10⁷ (99.50% inhibition), 1.78×10⁶ (99.98% inhibition) and 5.00×10⁴ (>99.99% inhibition) CFU/mL were recorded, respectively. It could thus be hypothesised that a systematic decrease in the adhesion of E. coli L1 cells was observed with an increase in the concentration of the SB24 crude extracts used to pre-treat the pegs of the lid on the MBEC™ assay.

The ability of the ST34 and SB12 biosurfactant crude extracts to inhibit adhesion of E. coli L1 is presented in FIGS. 1B-C. For the untreated pegs, an average of 7.54×10⁷ CFU/mL E. coli L1 cells was enumerated in the biofilm suspension using culture-based analysis. For the pegs treated with the ST34 biosurfactant crude extracts at concentrations of 6.25, 12.5, 25 and 50 mg/mL, culture based analysis of the E. coli L1 cells in the biofilm suspension indicated that 2.13×10⁷ (97.17% inhibition), 1.05×10⁷ (98.61% inhibition), 4.21×10⁶ (99.44% inhibition) and 3.33×10⁶ (99.93% inhibition) CFU/mL were recorded, respectively. For the pegs treated with SB12 biosurfactant crude extracts at concentrations of 6.25, 12.5, 25 and 50 mg/mL, culture based analysis of the E. coli L1 cells in the biofilm suspension indicated that 7.17×10² (>99.99% inhibition), 5.50×10² (>99.99% inhibition), 3.88×10² (>99.99% inhibition) and 2.50×10² (>99.99% inhibition) CFU/mL were recorded, respectively. While a decrease in the adhesion of E. coli L1 cells was observed with an increase in the concentration of the ST34 biosurfactant crude extracts used to pre-treat the pegs of the lid on the MBEC™ assay, a similar and significant decrease in E. coli CFU/mL was observed for the various concentrations of SB12 used to pre-treat the pegs.

In addition to the culturing analysis, the potential of the biosurfactant crude extract obtained from SB24 to inhibit biofilm formation by E. coli L1 was also evaluated using EMA-qPCR (FIG. 1D). A qPCR efficiency of 2.17 (109%) was obtained, with a linear regression coefficient (R²) value of 0.99 recorded for the standard curve. Using the standard curve, viable E. coli L1 gene copy numbers were quantified in the biofilm suspension obtained for the untreated and corresponding biosurfactant crude extract pre-treated pegs of the MBEC™ assay at various concentrations and are presented as uidA gene copies per mL (FIGS. 1D-F). For the untreated pegs, an average of 2.99×10⁹ gene copies/mL was enumerated in the biofilm suspension using EMA-qPCR analysis (FIG. 1D). For the pegs treated with the SB24 biosurfactant crude extracts at concentrations of 6.25, 12.5, 25 and 50 mg/mL, EMA-qPCR analysis of the E. coli L1 gene copies in the biofilm suspension indicated that 3.53×10⁸ (88.19% inhibition), 1.67×10⁸ (94.40% inhibition), 2.35×10⁷ (99.21% inhibition) and 3.62×10⁶ (99.88% inhibition) uidA gene copies/mL were recorded, respectively. The adhesion of E. coli L1 cells thus decreased with an increase in the concentration of the SB24 crude extracts used to pre-treat the pegs of the lid on the MBEC™ assay.

The ability of the ST34 and SB12 biosurfactant crude extracts to inhibit adhesion of E. coli L1 is presented in FIGS. 1E-F. For the untreated pegs, an average of 2.99×10⁹ gene copies/mL E. coli L1 cells was enumerated in the biofilm suspension using EMA-qPCR analysis (FIGS. 1E-F). For the pegs treated with the ST34 biosurfactant crude extracts at concentrations of 6.25, 12.5, 25 and 50 mg/mL, EMA-qPCR analysis of the E. coli L1 cells in the biofilm suspension indicated that 3.55×10⁷ (98.81% inhibition), 7.71×10⁶ (99.74% inhibition), 4.44×10⁶ (99.85% inhibition) and 3.05×10⁶ (99.90% inhibition) uidA gene copies/mL were recorded, respectively. For the pegs treated with the SB12 biosurfactant crude extracts at concentrations of 6.25, 12.5, 25 and 50 mg/mL, EMA-qPCR analysis of the E. coli L1 cells in the biofilm suspension indicated that 2.17×10⁸ (92.73% inhibition), 1.85×10⁸ (93.82% inhibition), 7.15×10⁷ (97.61% inhibition) and 6.60×10⁶ (99.78% inhibition) gene copies/mL were recorded, respectively. Similarly to the results obtained for the SB24 crude extract, a decrease in the adhesion of E. coli L1 cells was observed with an increase in the concentration of the ST34 and SB12 biosurfactant crude extracts used to pre-treat the pegs of the lid on the MBEC™ assay.

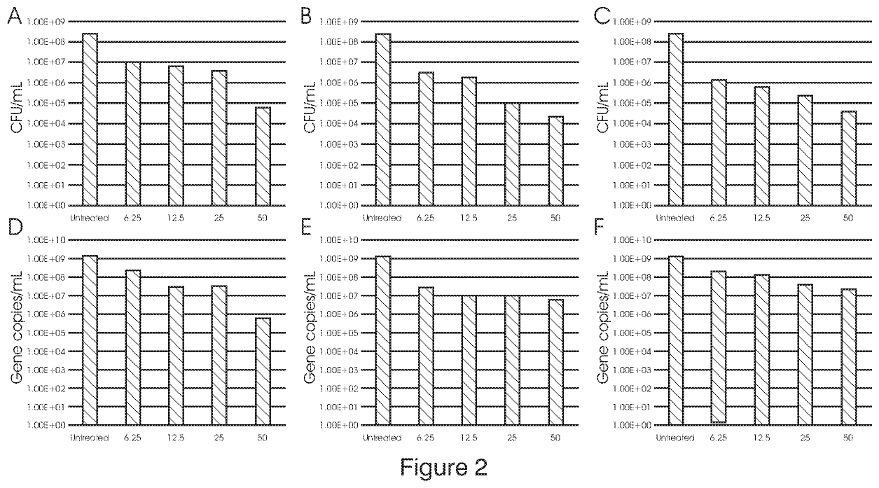

FIG. 2 illustrates a reduction in adhesion of L. monocytogenes C1 to surfaces of the MBEC™ assay precoated with biosurfactant crude extracts SB24, ST34 and SB12 at various concentrations (6.25, 12.5, 25 and 50 mg/mL). The L. monocytogenes cells capable of attaching to the coated surfaces were quantified using plate counts (culturing analysis) and EMA-qPCR analysis (an untreated control was included for each biosurfactant). The plate counts of L. monocytogenes C1 are presented in; (A) SB24, (B) ST34 and (C) SB12. The EMA-qPCR analysis results of L. monocytogenes C1 are presented in; (D) SB24, (E) ST34 and (F) SB12.

Referring to FIG. 2, the ability of the SB24 biosurfactant crude extract to inhibit adhesion of L. monocytogenes C1 cells is presented in FIG. 2A. For the untreated pegs, an average of 2.74×10⁸ CFU/mL L. monocytogenes C1 cells was enumerated in the biofilm suspension using culture-based analysis. For the pegs treated with the SB24 biosurfactant crude extracts at concentrations of 6.25, 12.5, 25 and 50 mg/mL, culture based analysis of the L. monocytogenes C1 cells in the biofilm suspension indicated that 9.75×10⁶ (96.44% inhibition), 7.46×10⁶ (97.28% inhibition), 4.33×10⁶ (98.42% inhibition) and 7.09×10⁴ (99.97% inhibition) CFU/mL were recorded, respectively. A significant decrease in the adhesion of L. monocytogenes C1 cells was thus observed at the highest concentration (50 mg/mL) of the SB24 crude extract used to pre-treat the pegs of the lid on the MBEC™ assay.

The ability of the ST34 and SB12 biosurfactant crude extracts to inhibit adhesion of L. monocytogenes C1 cells is presented in FIGS. 2B-C. For the untreated pegs, an average of 2.74×10⁸ CFU/mL L. monocytogenes C1 cells was enumerated in the biofilm suspension using culture-based analysis. For the pegs treated with the ST34 biosurfactant crude extracts at concentrations of 6.25, 12.5, 25 and 50 mg/mL, culture based analysis of the L. monocytogenes C1 cells in the biofilm suspension indicated that 3.84×10⁶ (98.60% inhibition), 1.82×10⁶ (99.33% inhibition), 1.10×10⁵ (99.96% inhibition) and 2.73×10⁴ (99.99% inhibition) CFU/mL were recorded, respectively. Similarly, for the pegs treated with SB12 biosurfactant crude extracts at concentrations of 6.25, 12.5, 25 and 50 mg/mL, culture based analysis of the L. monocytogenes C1 cells in the biofilm suspension indicated that 1.32×10⁶ (99.52% inhibition), 8.14×10⁵ (99.70% inhibition), 2.42×10⁵ (99.91% inhibition) and 4.85×10⁴ (99.98% inhibition) CFU/mL were recorded, respectively. For both ST34 and SB12, a significant decrease in the adhesion of L. monocytogenes C1 CFU/mL was thus observed at the highest concentration (50 mg/mL) of the biosurfactant crude extracts used to pre-treat the pegs of the lid on the MBEC™ assay.

For the EMA-qPCR analysis, using the standard curve, viable L. monocytogenes C1 gene copy numbers were quantified in the biofilm suspension obtained for the untreated and corresponding biosurfactant crude extract pre-treated pegs of the MBEC™ assay at various concentrations and are presented as prfA gene copies per mL (FIGS. 2D-F). A qPCR efficiency of 1.92 (96%) was obtained, with a linear regression coefficient (R²) value of 1.0. recorded for the standard curve. The ability of the SB24 biosurfactant crude extract to inhibit adhesion of L. monocytogenes C1 is presented in FIG. 2D. For the untreated pegs, an average of 1.34×10⁹ gene copies/mL L. monocytogenes C1 cells were enumerated in the biofilm suspension using EMA-qPCR analysis (FIG. 2D). For the pegs treated with the SB24 biosurfactant crude extracts at concentrations of 6.25, 12.5, 25 and 50 mg/mL, EMA-qPCR analysis of the L. monocytogenes C1 cells in the biofilm suspension indicated that 2.37×10⁸ (82.36% inhibition), 3.48×10⁷ (97.41% inhibition), 3.44×10⁷ (97.43% inhibition) and 6.42×10⁵ (99.95% inhibition) gene copies/mL were recorded, respectively. It could thus be hypothesised that a systematic decrease in the adhesion of L. monocytogenes C1 cells was observed with an increase in the concentration of the SB24 crude extracts from 6.25 mg/mL to 12.5 mg/mL, with a significant reduction recorded when 50 mg/mL was used to pre-treat the pegs of the lid on the MBEC™ assay.

The ability of the ST34 and SB12 biosurfactant crude extracts to inhibit adhesion of L. monocytogenes C1 is presented in FIGS. 2E-F. For the untreated pegs, an average of 1.34×10⁹ gene copies/mL L. monocytogenes C1 cells was enumerated in the biofilm suspension using EMA-qPCR analysis (FIGS. 2E-F). For the pegs treated with the ST34 biosurfactant crude extracts at concentrations of 6.25, 12.5, 25 and 50 mg/mL, EMA-qPCR analysis of the L. monocytogenes C1 cells in the biofilm suspension indicated that 3.14×10⁷ (97.66% inhibition), 1.10×10⁷ (99.18% inhibition), 1.25×10⁷ (99.07% inhibition) and 6.83×10⁶ (99.49% inhibition) prfA gene copies/mL were recorded, respectively. For the pegs treated with the SB12 biosurfactant crude extracts at concentrations of 6.25, 12.5, 25 and 50 mg/mL, EMA-qPCR analysis of the L. monocytogenes C1 cells in the biofilm suspension indicated that 2.52×10⁸ (81.23% inhibition), 1.25×10⁸ (90.71% inhibition), 4.39×10⁷ (96.73% inhibition) and 2.55×10⁷ (98.10% inhibition) gene copies/mL were recorded, respectively. A similar decrease in the adhesion of L. monocytogenes C1 cells was thus observed with an increase in the concentration of the ST34 and SB12 biosurfactant crude extracts used to pre-treat the pegs of the lid on the MBEC™ assay.

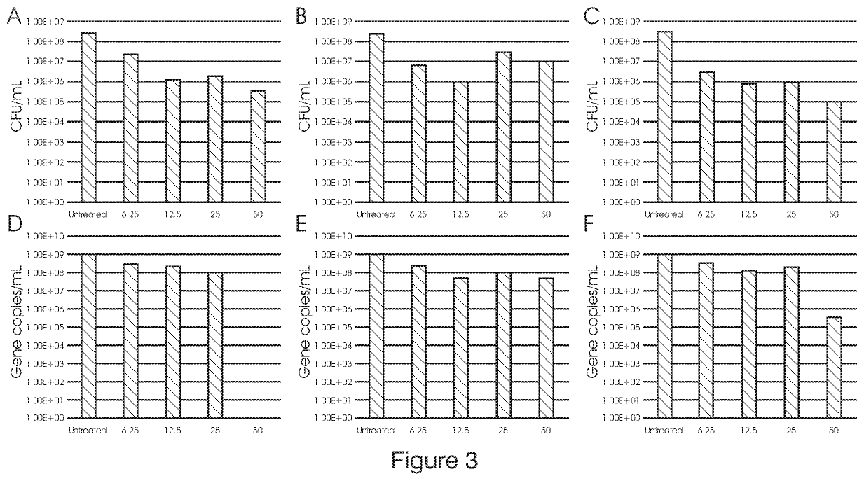

FIG. 3 illustrates a reduction in adhesion of C. neoformans CAB 1055 to surfaces of the MBEC™ assay precoated with biosurfactant crude extracts SB24, ST34 and SB12 at various concentrations (6.25, 12.5, 25 and 50 mg/mL). The C. neoformans cells capable of attaching to the coated surfaces were quantified using plate counts (culturing analysis) and EMA-qPCR analysis (an untreated control was included for each biosurfactant). The plate counts of C. neoformans CAB 1055 are presented in; (A) SB24, (B) ST34 and (C) SB12. The EMA-qPCR analysis results of C. neoformans CAB 1055 are presented in; (D) SB24, (E) ST34 and (F) SB12.

Referring to FIG. 3, the ability of the SB24 biosurfactant crude extract to inhibit adhesion of C. neoformans CAB 1055 is presented in FIG. 3A. For the untreated pegs, an average of 2.81×10⁸ CFU/mL C. neoformans CAB 1055 cells was enumerated in the biofilm suspension using culture-based analysis. For the pegs treated with the SB24 biosurfactant crude extracts at concentrations of 6.25, 12.5, 25 and 50 mg/mL, culture based analysis of the C. neoformans CAB 1055 cells in the biofilm suspension indicated that 2.48×10⁷ (91.17% inhibition), 1.16×10⁶ (99.59% inhibition), 1.88×10⁶ (99.33% inhibition) and 3.75×10⁵ (99.87% inhibition) CFU/mL were recorded, respectively.

Fluctuations in the C. neoformans CAB 1055 counts were thus observed with an increase in the concentration of the SB24 crude extracts, with the greatest decrease observed when a concentration of 50 mg/mL was used to pre-treat the pegs of the lid on the MBEC™ assay.

The ability of the ST34 and SB12 biosurfactant crude extracts to inhibit adhesion of C. neoformans CAB 1055 is presented in FIGS. 3B-C. For the untreated pegs, an average of 2.81×10⁸ CFU/mL C. neoformans CAB 1055 cells was enumerated in the biofilm suspension using culture-based analysis. For the pegs treated with the ST34 biosurfactant crude extracts at concentrations of 6.25, 12.5, 25 and 50 mg/mL, culture based analysis of the C. neoformans CAB 1055 cells in the biofilm suspension indicated that 7.06×10⁶ (97.49% inhibition), 9.08×10⁵ (99.68% inhibition), 2.95×10⁷ (89.50% inhibition) and 9.70×10⁶ (96.55% inhibition) CFU/mL were recorded, respectively. For the pegs treated with SB12 biosurfactant crude extracts at concentrations of 6.25, 12.5, 25 and 50 mg/mL, culture based analysis of the C. neoformans CAB 1055 cells in the biofilm suspension indicated that 2.95×10⁶ (98.95% inhibition), 7.06×10⁵ (99.75% inhibition), 9.70×10⁵ (99.65% inhibition) and 9.08×10⁴ (99.97% inhibition) CFU/mL were recorded, respectively. Fluctuations in the C. neoformans CAB 1055 counts were thus observed with an increase in the concentration of ST34 and SB12 biosurfactant crude extracts, with the highest reductions observed at the concentrations of 12.5 (99.68%) and 50 mg/mL (99.97%), respectively.

For the EMA-qPCR, using the standard curve, viable C. neoformans CAB 1055 gene copy numbers were quantified in the biofilm suspension obtained for the untreated and corresponding biosurfactant crude extract pre-treated pegs of the MBEC™ assay at various concentrations and are presented as 5.8S rDNA gene copies per mL (FIGS. 3D-F). A qPCR efficiency of 2.17 (109%) was obtained, with a linear regression coefficient (R²) value of 0.99 recorded for the standard curve. For the untreated pegs, an average of 1.18×10⁹ gene copies/mL C. neoformans CAB 1055 cells was enumerated in the biofilm suspension using EMA-qPCR analysis (FIG. 3D). For the pegs treated with the SB24 biosurfactant crude extracts at concentrations of 6.25, 12.5 and 25 mg/mL, EMA-qPCR analysis of the C. neoformans CAB 1055 cells in the biofilm suspension indicated that 3.70×10$ (68.71% inhibition), 2.51×10⁸ (78.74% inhibition) and 1.14×10⁸ (90.33% inhibition) gene copies/mL were recorded, respectively. A lower limit of detection (LLOD<6 gene copies/mL) was however recorded after the C. neoformans CAB 1055 was exposed to the 50 mg/mL of the SB24 crude extracts. It could thus be hypothesised that a systematic decrease in the adhesion of C. neoformans CAB 1055 cells was observed with an increase in the concentration (6.25 to 25 mg/mL) of the SB24 crude extracts used to pre-treat the pegs of the lid on the MBEC™ assay.

The ability of the ST34 and SB12 biosurfactant crude extracts to inhibit adhesion of C. neoformans CAB 1055 is presented in FIGS. 3E-F. For the untreated pegs, an average of 1.18×10⁹ gene copies/mL C. neoformans CAB 1055 cells was enumerated in the biofilm suspension using EMA-qPCR analysis. For the pegs treated with the ST34 biosurfactant crude extracts at concentrations of 6.25, 12.5, 25 and 50 mg/mL, EMA-qPCR analysis of the C. neoformans CAB 1055 cells in the biofilm suspension indicated that 2.70×10⁸ (77.15% inhibition), 6.11×10⁷ (94.83% inhibition), 1.18×10⁸ (90.02% inhibition) and 5.66×10⁷ (95.21% inhibition) 5.8S rDNA gene copies/mL were recorded, respectively (FIG. 3E). For the pegs treated with the SB12 biosurfactant crude extracts at concentrations of 6.25, 12.5, 25 and 50 mg/mL, EMA-qPCR analysis of the C. neoformans CAB 1055 cells in the biofilm suspension indicated that 3.70×10⁸ (68.67% inhibition), 1.35×10⁸ (88.58% inhibition), 2.10×10⁸ (82.21% inhibition) and 3.85×10⁵ (99.97% inhibition) gene copies/mL were recorded, respectively (FIG. 3F). Fluctuations in the C. neoformans CAB 1055 gene copies were thus observed with an increase in the concentration of ST34 and SB12, with the highest reduction of 95.21% and 99.97% observed at the concentration of 50 mg/mL, respectively, for both biosurfactant crude extracts.

Overall, based on the results of the plate counts and EMA-qPCR analysis, the crude extracts obtained from the P. aeruginosa SB24 and B. amyloliquefaciens SB12 and ST34 displayed the greatest inhibition of the E. coli L1, C. neoformans CAB 1055 and L. monocytogenes C1 cells to form a biofilm on the pegs at 50 mg/mL. Based on the EMA-qPCR analysis, intact cells were however, still present at the highest concentration of SB24, ST34 and SB12 analysed. The overall reduction of the microbial cells attaching to the biosurfactant pre-treated pegs (in comparison to the untreated pegs) may be attributed to the presence of the biosurfactants that may display a broad-spectrum antimicrobial activity against various microorganisms including E. coli, L. monocytogenes and C. neoformans strains. As previously mentioned, the ST34 and SB12 crude extracts were shown to contain various analogues of surfactin and bacillomycin, which may have synergistic antimicrobial activity against Gram-negative bacteria and fungal strains.

Without commitment to the veracity thereof, it is hypothesised that the biosurfactant extracts [SB24 (composed of rhamnolipid congeners and homologues) and ST34 and SB12 (composed of surfactin and bacillomycin analogues and homologues)] reduced microbial cell attachment by modifying the hydrophobicity of the coated materials, which interfered with microbial adhesion and desorption processes.

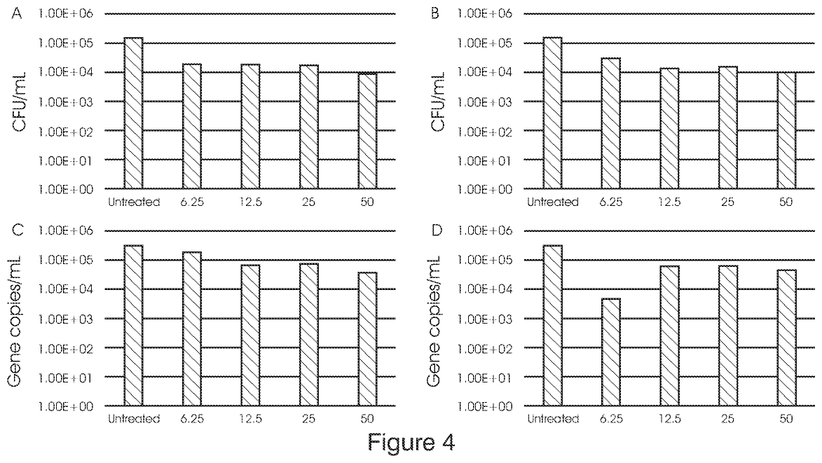

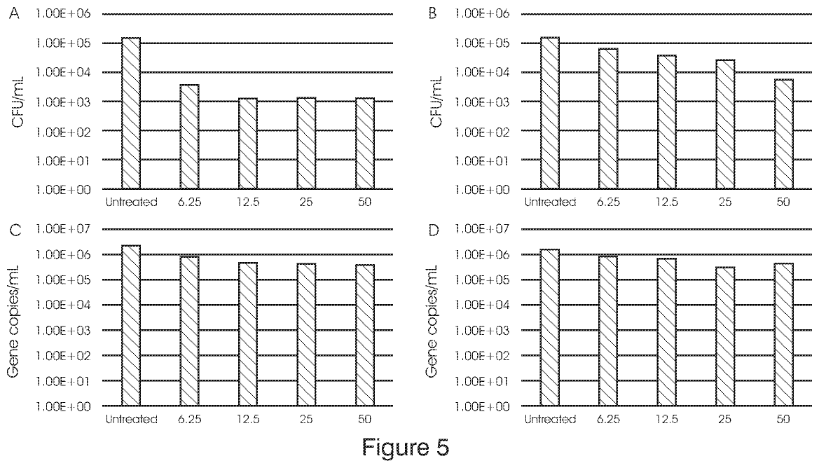

2.1.2. Biosurfactant Crude Extracts Obtained from S. marcescens Strains (FIGS. 4-5)

Standard culture-based methods and EMA-qPCR analysis were used to evaluate the potential of the biosurfactant crude extracts produced by the S. marcescens P1 and NP1 strains to inhibit biofilm formation of P. aeruginosa S1 68 and E. faecalis S1. The crude biosurfactant extracts were used for the pre-treatment of the surface of the pegs of the MBEC™ assay, at concentration ranges of 6.25 to 50 mg/mL.

FIG. 4 illustrates a reduction in adhesion of P. aeruginosa S1 68 to surfaces of the MBEC™ assay precoated with biosurfactant crude extracts P1 and NP1 at various concentrations (6.25, 12.5, 25 and 50 mg/mL). The P. aeruginosa cells capable of attaching to the coated surfaces were quantified using plate counts (culturing analysis) and EMA-qPCR analysis (an untreated control was included for each biosurfactant). The plate counts of P. aeruginosa S1 68 are presented in; (A) P1 and (B) NP1. The EMA-qPCR analysis of P. aeruginosa S1 68 are presented in; (C) P1 and (D) NP1.

Referring to FIG. 4, the ability of the P1 and NP1 biosurfactant crude extracts to inhibit adhesion of P. aeruginosa S1 68 is presented in FIGS. 4A-D. For the uncoated pegs, an average of 1.49×10⁵ CFU/mL was enumerated in the biofilm suspension using culture-based analysis (FIGS. 4A-B). For the pegs treated with the P1 crude extract at concentrations of 6.25, 12.5, 25 and 50 mg/mL, the culture based analysis of the P. aeruginosa S1 68 cells in the biofilm suspension indicated that 1.80×10⁴ CFU/mL (87.89% inhibition), 1.80×10⁴ CFU/mL (87.89% inhibition), 1.63×10⁴ CFU/mL (89.01% inhibition) and 8.33×10³ CFU/mL (94.39% inhibition) were recorded, respectively (FIG. 4A). For the pegs treated with the NP1 crude extract at concentrations of 6.25, 12.5, 25 and 50 mg/mL, the culture-based analysis of the P. aeruginosa S1 68 cells in the biofilm suspension indicated that 2.73×10⁴ CFU/mL (81.67% inhibition), 1.38×10⁴ CFU/mL (90.75% inhibition), 1.40×10⁴ CFU/mL (90.58% inhibition) and 1.03×10⁴ CFU/mL (93.11% inhibition) were recorded, respectively (FIG. 4B). It could thus be hypothesised that a similar decrease in the adhesion of P. aeruginosa S1 68 cells was observed with an increase in the concentration of the P1 and NP1 crude extracts used to pre-treat the pegs of the lid on the MBEC™ assay.

In addition to the culturing analysis, the potential of the biosurfactant crude extracts obtained from P1 and NP1 to inhibit biofilm formation by P. aeruginosa S1 68 was evaluated using EMA-qPCR (FIGS. 4C-D). A qPCR efficiency of 1.96 (98%) was obtained, with a linear regression coefficient (R²) value of 0.98 recorded for the standard curve. Using the standard curve, P. aeruginosa S1 68 gene copy numbers from intact cells were quantified in the biofilm suspension obtained for the uncoated and corresponding biosurfactant crude extract pre-treated pegs of the MBEC™ assay at various concentrations and are presented as oprl gene copies per mL. For the uncoated pegs, an average of 3.39×10⁵ gene copies/mL was enumerated in the biofilm suspension using EMA-qPCR analysis (FIGS. 4C-D).

For the pegs treated with the P1 crude extract at concentrations of 6.25, 12.5, 25 and 50 mg/mL, the EMA-qPCR analysis of the P. aeruginosa S1 68 cells in the biofilm suspension indicated that 1.98×10⁵ gene copies/mL (41.64% inhibition), 6.64×10⁴ gene copies/mL (80.41% inhibition), 7.30×10⁴ gene copies/mL (78.47% inhibition) and 3.57×10⁴ gene copies/mL (89.48% inhibition) were recorded, respectively (FIG. 4C). For the pegs treated with the NP1 crude extract at concentrations of 6.25, 12.5, 25 and 50 mg/mL, the EMA-qPCR analysis of the P. aeruginosa S1 68 cells in the biofilm suspension indicated that 4.83×10³ gene copies/mL (98.85% inhibition), 6.62×10⁴ gene copies/mL (80.46% inhibition), 6.64×10⁴ gene copies/mL (80.41% inhibition) and 4.96×10⁴ gene copies/mL (85.35% inhibition) were recorded, respectively (FIG. 4D). Fluctuations in the oprl gene copies were thus observed with an increase in the concentration of P1 and NP1 biosurfactant crude extracts, with the greatest inhibition of P. aeruginosa S1 68 biofilm formation for the P1 crude extract observed at 50 mg/mL, while the highest reduction for the NP1 crude extract was observed at a concentration of 6.25 mg/L.

FIG. 5 illustrates a reduction in adhesion of E. faecalis S1 to surfaces of the MBEC™ assay precoated with biosurfactant crude extracts P1 and NP1 at various concentrations (6.25, 12.5, 25 and 50 mg/mL). The E. faecalis S1 cells capable of attaching to the coated surfaces were quantified using plate counts (culturing analysis) and EMA-qPCR analysis (an untreated control was included for each biosurfactant). The plate counts of E. faecalis S1 are presented in; (A) P1 and (B) NP1. The EMA-qPCR analysis of E. faecalis S1 are presented in; (C) P1 and (D) NP1.

Referring to FIG. 5, FIGS. 5A-D illustrate the ability of the P1 and NP1 biosurfactant crude extract to inhibit adhesion of E. faecalis S1. For the uncoated pegs, an average of 1.40×10⁵ CFU/mL was enumerated in the biofilm suspension using culture-based analysis (FIGS. 5A-B). For the pegs treated with the P1 crude extract at concentrations of 6.25, 12.5, 25 and 50 mg/mL, the culture based analysis of the E. faecalis S1 cells in the biofilm suspension indicated that 3.80×10³ CFU/mL (97.29% inhibition), 1.30×10³ CFU/mL (99.07% inhibition), 1.30×10³ CFU/mL (99.07% inhibition) and 1.30×10³ CFU/mL (99.07% inhibition) were recorded, respectively (FIG. 5A). For the pegs treated with the NP1 crude extract at concentrations of 6.25, 12.5, 25 and 50 mg/mL, the culture-based analysis of the E. faecalis S1 cells in the biofilm suspension indicated that 6.07×10⁴ CFU/mL (55.67% inhibition), 3.47×10⁴ CFU/mL (75.24% inhibition), 2.67×10⁴ CFU/mL (80.95% inhibition) and 5.83×10³ CFU/mL (95.83% inhibition) were recorded, respectively (FIG. 5B). While a similar decrease in the adhesion of E. faecalis S1 cells was obtained when the pegs of the lid on the MBEC™ assay were pre-treated with the various concentrations of P1, a significant decrease was obtained for the 50 mg/mL NP1 crude extracts.

In addition to the culturing analysis, the potential of the biosurfactant crude extracts obtained from P1 and NP1 to inhibit biofilm formation by E. faecalis S1 was evaluated using EMA-qPCR (FIGS. 5 C-D). A qPCR efficiency of 2.08 (104%) was obtained, with a linear regression coefficient (R²) value of 1.00 for the standard curve. Using the standard curve, E. faecalis S1 gene copy numbers from intact cells were quantified in the biofilm suspension obtained for the uncoated and corresponding biosurfactant crude extract pre-treated pegs of the MBEC™ assay at various concentrations and are presented as 23S rRNA gene copies per mL. For the uncoated pegs, an average of 1.48×10⁶ gene copies/mL was enumerated in the biofilm suspension using EMA-qPCR analysis (FIGS. 5C-D). For the pegs treated with the P1 crude extract at concentrations of 6.25, 12.5, 25 and 50 mg/mL, the EMA-qPCR analysis of the E. faecalis S1 cells in the biofilm suspension indicated that 8.93×10⁵ gene copies/mL (39.48% inhibition), 4.83×10⁵ gene copies/mL (67.29% inhibition), 4.45×10⁵ (69.83% inhibition) and 3.66×10⁵ gene copies/mL (75.20% inhibition) were recorded, respectively (FIG. 5C). For the pegs treated with the NP1 crude extract at concentrations of 6.25, 12.5, 25 and 50 mg/mL, the EMA-qPCR analysis of the E. faecalis S1 cells in the biofilm suspension indicated that 8.72×10⁵ gene copies/mL (40.90% inhibition), 6.85×10⁵ gene copies/mL (53.57% inhibition), 3.11×10⁵ gene copies/mL (78.91% inhibition) and 3.93×10⁵ gene copies/mL (73.35% inhibition) were recorded, respectively (FIG. 5D). Fluctuations in the 23S rRNA gene copies were thus observed with an increase in the concentration of P1 and NP1 biosurfactant crude extracts.

Overall, in comparison to the microbial cells attached to the untreated pegs of the MBEC™ assay, a decrease in the P. aeruginosa S1 68 and E. faecalis S1 colony forming units and the respective gene copy numbers was observed at all the concentrations of the P1 and NP1 crude extracts used for the pre-treatment of the pegs. However, based on the results of the plate counts and EMA-qPCR analysis, the crude extracts obtained from the S. marcescens P1 crude extract displayed the greatest inhibition (albeit not significantly) of P. aeruginosa S1 68 and E. faecalis S1 biofilm formation on the pegs at 50 mg/mL.

The crude extract obtained from S. marcescens P1 is understood to comprise serratamolides (lipopeptide), glucosamine derivatives and prodigiosin (pigment), while the crude extract obtained from S. marcescens NP1 is understood to comprise serratamolides (lipopeptide) and glucosamine derivatives (amino sugar). Although no studies have indicated that serratamolides or glucosamine derivatives inhibit or reduce microbial adhesion onto a surface, purified serratamolide homologues, prodigiosin and glucosamine derivatives display antimicrobial activity. In addition, a similar lipopeptide referred to as serrawettin W2, produced by Serratia sp., can inhibit the microbial adhesion of C. albicans to a polypropylene surface. It is therefore hypothesised (without commitment to the veracity thereof) that the reduction in the formation of P. aeruginosa S1 68 and E. faecalis S1 biofilms on the surface of the pegs precoated with P1 and NP1 may be attributed to a combination of serratamolides (P1 and NP1) and prodigiosin (P1) and glucosamine derivatives (P1 and NP1), respectively, present within the crude extracts. It is also hypothesised that the observed reduction in biofilm formation may have been due to a change in hydrophobicity of the pegs after absorption of the P1 and NP1 biosurfactant crude extracts onto the surface of the pegs.

### 3. IMMOBILISATION OF THE BIOSURFACTANTS ON THE MATERIALS

The steps described below were performed using crude extracts of the biosurfactants. However, it will be appreciated that the same steps may also be performed using compounds isolated from crude extracts, or a product resulting from treatment of a crude extract to obtain a purer fraction of the biosurfactant in question. Such treatment may, for example, include high performance liquid chromatography.

### 3.1. Biosurfactant Modification

This step involved chemical modification of the biosurfactants to enhance their attachment and immobilisation on the material surfaces by promoting covalent bonding of the biosurfactants to the surfaces.

Separate protocols were followed for the modification of the biosurfactants produced by the SB24, ST34 and SB12 strains on the one hand, and those produced by the P1 and NP1 strains on the other hand.

### 3.1.1. Modification of Biosurfactants Produced by SB24, ST34 and SB12

The biosurfactant crude extracts obtained from SB24, ST34 and SB12 were modified as illustrated in Scheme 1 below. The rhamnolipids, surfactin and bacillomycin biosurfactant compounds produced by these strains contain carboxylic groups, which may be functionalised by generating activated ester in the presence of N-Hydroxysuccinimide (NHS) under anhydrous mild Steglich esterification reaction conditions.

For the modification of the crude extracts obtained from SB24, a 100 mL round bottom flask was charged with SB24 biosurfactant crude extract (350 mg, 0.7 mmol), N,N′-Dicyclohexylcarbodiimide (DCC, 166.1 mg, 0.805 mmol), 4-Dimethylaminopyridine (DMAP, 9.41 mg, 0.077 mmol), N-Hydroxysuccinimide (NHS, 402.8 mg, 3.5 mmol) and dry ethanol and stirred overnight at room temperature.

For the modification of the crude extracts obtained from ST34 and SB12, a 350 mg (0.35 mmol) quantity of each biosurfactant extract, DCC (83.1 mg, 0.402 mmol), DMAP (4.7 mg, 0.0385 mmol) and NHS (201.4 mg, 1.75 mmol) were added into respective 100 mL round bottom flasks and were solubilised in dry ethanol. Thereafter, the reaction mixture was concentrated in a rotary evaporator and washed with ethanol yielding the modified biosurfactant crude extract.

The coupling of the carboxylic acid group to N-hydroxysuccinimide (NHS) in each case provides a more reactive moiety that can readily react with terminal amine groups on the APTES linker that is used for the covalent bonding step (see below). Carbonyl groups can react with amine groups to form imine, which is an acid labile moiety that is stable under non-hydrolysing conditions.

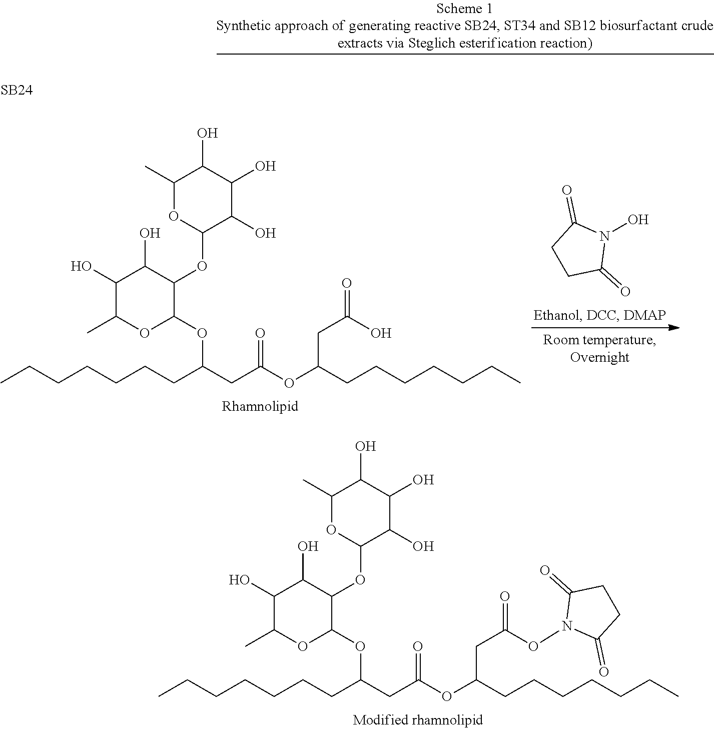

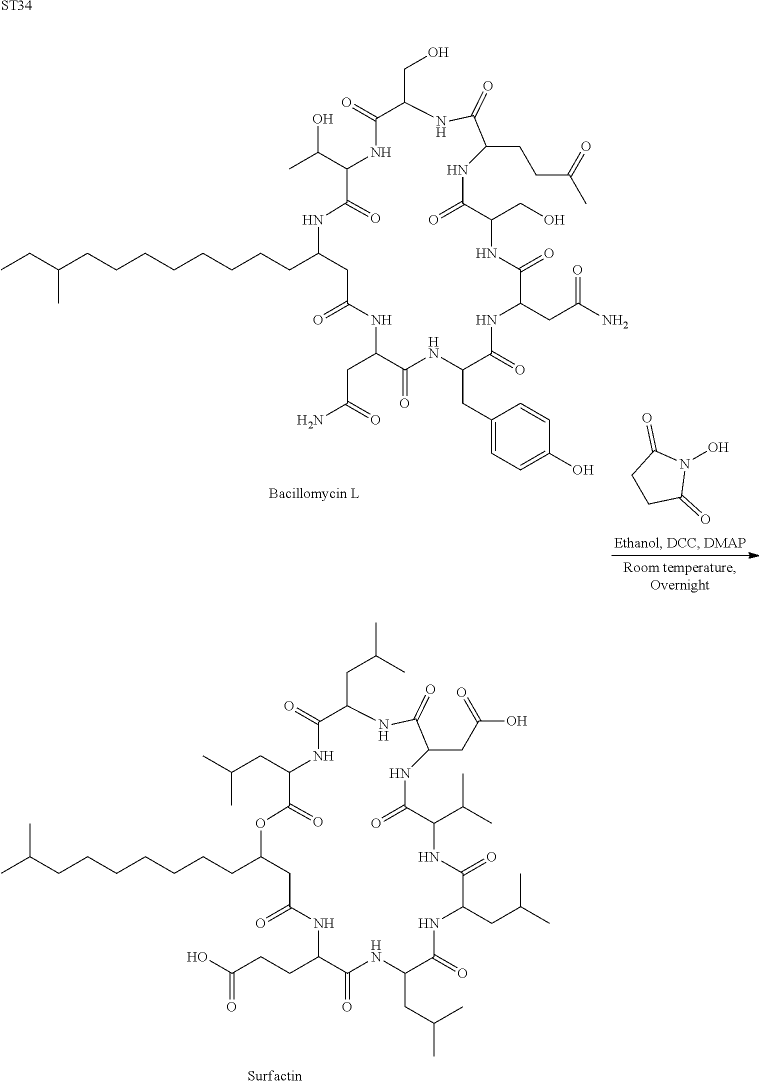 [SMILES: "ST34" ; CCC(C)CCCCCCCCC1CC(=O)NC(CC(N)=O)C(=O)NC(Cc2ccc(O)cc2)C(=O)NC(CC(N)=O)C(=O)NC(CO)C(=O)NC(CCC(C)=O)C(=O)NC(CO)C(=O)NC(C(C)O)C(=O)N1 ; O=C1CCC(=O)N1O ; CC(C)CCCCCCCC1CC(=O)NC(CCC(=O)O)C(=O)NC(CC(C)C)C(=O)NC(CC(C)C)C(=O)NC(C(C)C)C(=O)NC(CC(=O)O)C(=O)NC(CC(C)C)C(=O)NC(CC(C)C)C(=O)O1]

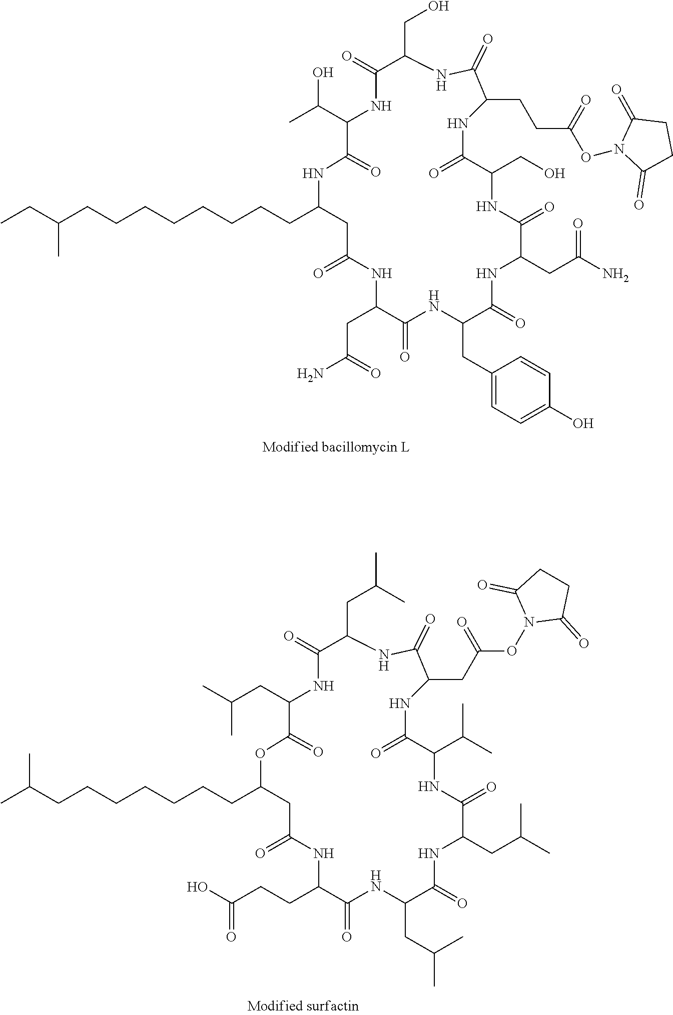 [SMILES: CCC(C)CCCCCCCCC1CC(=O)NC(CC(N)=O)C(=O)NC(Cc2ccc(O)cc2)C(=O)NC(CC(N)=O)C(=O)NC(CO)C(=O)NC(CCC(=O)ON2C(=O)CCC2=O)C(=O)NC(CO)C(=O)NC(C(C)O)C(=O)N1 ; CC(C)CCCCCCCC1CC(=O)NC(CCC(=O)O)C(=O)NC(CC(C)C)C(=O)NC(CC(C)C)C(=O)NC(C(C)C)C(=O)NC(CC(=O)ON2C(=O)CCC2=O)C(=O)NC(CC(C)C)C(=O)NC(CC(C)C)C(=O)O1]

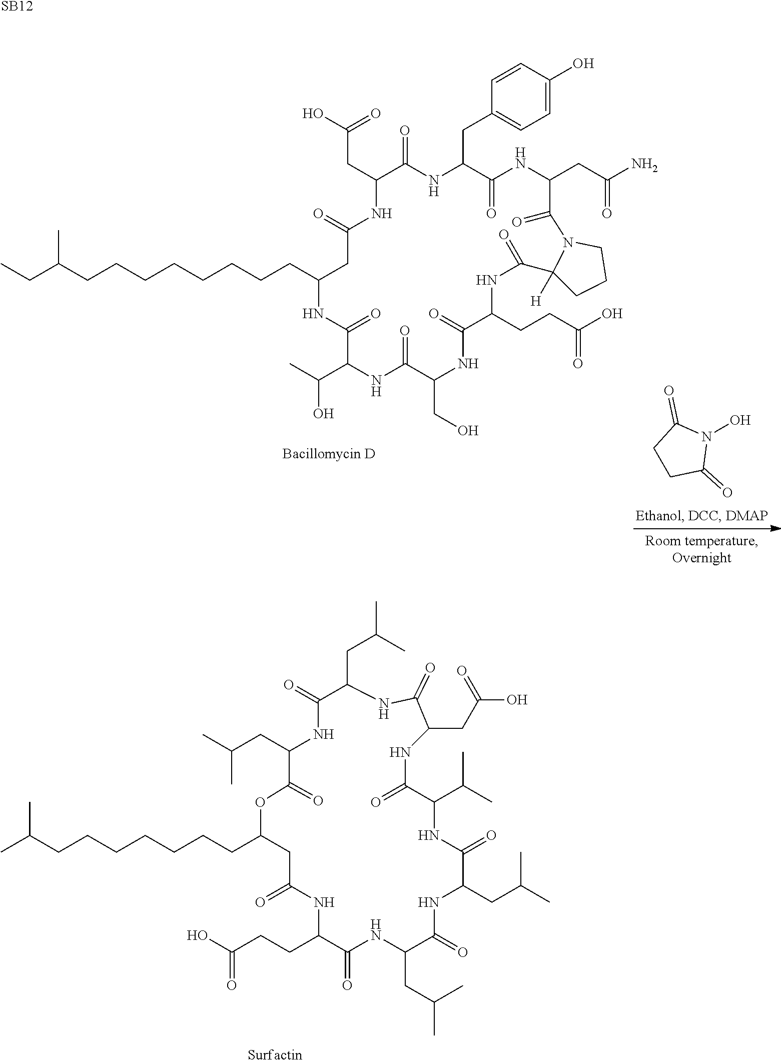 [SMILES: "SB12" ; CCC(C)CCCCCCCCC1CC(=O)NC(CC(=O)O)C(=O)NC(Cc2ccc(O)cc2)C(=O)NC(CC(N)=O)C(=O)N2CCCC2C(=O)NC(CCC(=O)O)C(=O)NC(CO)C(=O)NC(C(C)O)C(=O)N1 ; O=C1CCC(=O)N1O ; CC(C)CCCCCCCC1CC(=O)NC(CCC(=O)O)C(=O)NC(CC(C)C)C(=O)NC(CC(C)C)C(=O)NC(C(C)C)C(=O)NC(CC(=O)O)C(=O)NC(CC(C)C)C(=O)NC(CC(C)C)C(=O)O1]

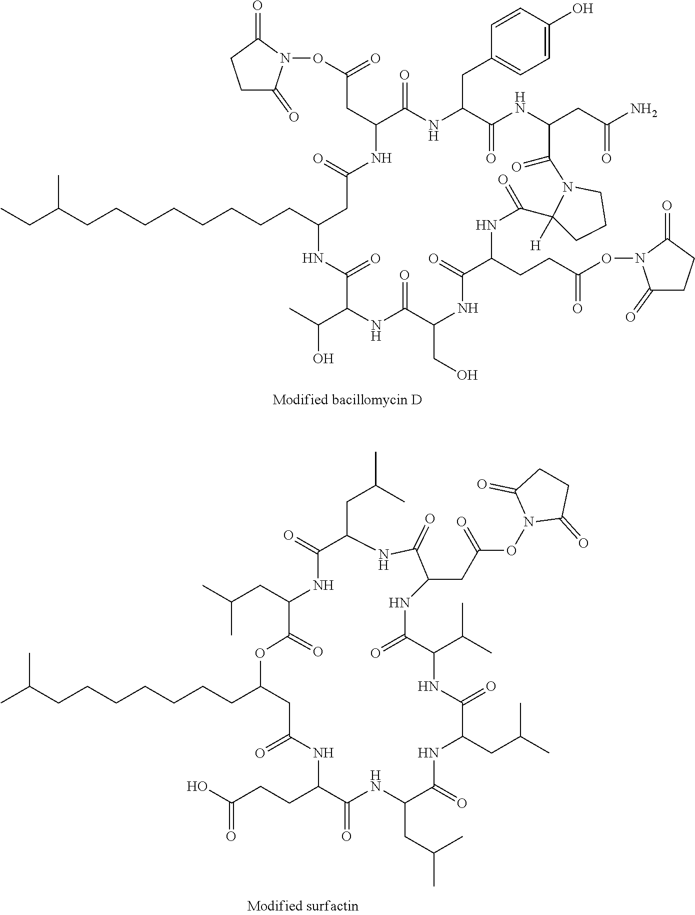 [SMILES: CCC(C)CCCCCCCCC1CC(=O)NC(CC(=O)ON2C(=O)CCC2=O)C(=O)NC(Cc2ccc(O)cc2)C(=O)NC(CC(N)=O)C(=O)N2CCCC2C(=O)NC(CCC(=O)ON2C(=O)CCC2=O)C(=O)NC(CO)C(=O)NC(C(C)O)C(=O)N1 ; CC(C)CCCCCCCC1CC(=O)NC(CCC(=O)O)C(=O)NC(CC(C)C)C(=O)NC(CC(C)C)C(=O)NC(C(C)C)C(=O)NC(CC(=O)ON2C(=O)CCC2=O)C(=O)NC(CC(C)C)C(=O)NC(CC(C)C)C(=O)O1]

### 3.1.2. Modification of Biosurfactants Produced by P1 and NP1 Strains

The biosurfactant crude extracts obtained from P1 and NP1 were modified as illustrated in Scheme 2 below. The serrawettin W1 and glucosamine derivatives produced by P1 and NP1 contain hydroxyl groups (—OH) which can be functionalised by replacing them with chlorine groups (—Cl). This method involves solubilising the biosurfactant and treating it with thionyl chloride and pyridine.

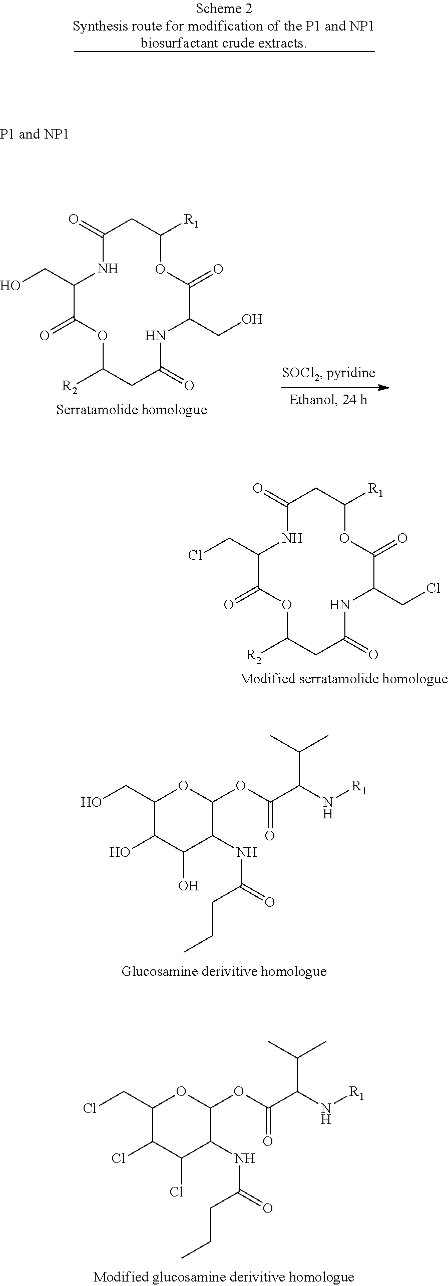

By way of example, P1 and NP1 biosurfactant crude extracts (350 mg, 0.714 mmol), pyridine (282.47 mg, 3.571 mmol) and dry ethanol were added into respective round bottom flasks (100 mL), each equipped with a magnetic stirrer, and chilled. Following solubilisation of the respective compounds, nitrogen gas was bubbled into the solutions. Thionyl chloride (424.8 mg, 3.571 mmol) was slowly added dropwise into the mixture and the reaction was continuously stirred for 20 h at ambient temperature. Subsequently, the excess solvent was removed under reduced pressure and the residue was washed three times with dichloromethane. Following the washing step, the mixture (composed of the modified biosurfactant compounds) was placed in a 40° C. oven for approximately 4 hrs to dry. All modified biosurfactant compounds were sealed in inert vials and stored at −20° C. until required.

The modifications to the biosurfactants were confirmed by Attenuated Total Reflectance-Fourier Transform Infrared (ATR-FTIR) analysis.

The modifications were also confirmed using Ultra Performance Liquid Chromatography coupled to Electrospray Ionisation Mass Spectrometry (UPLC-ESI-MS) analysis. Approximately 200 μg of the dry chemically modified biosurfactant crude extracts and 200 μg of dry unmodified biosurfactant crude extracts were dissolved in 40% acetonitrile and were analysed using the UPLC-ESI-MS.

Although the modification methods described above are preferred, it will be appreciated that other methods are available for modifying the biosurfactants to promote their reactivity. These other methods also fall within the scope of the invention. The serrawettins, for example, may be modified by oxidation to aldehydes or carboxylic acids. Modifications of that type may promote reactivity of the serrawettins with the silane linker. If, for example, the chosen silane linker has thiol or carboxyl functionalities, this type of modification may promote Michael-type reactions or esterification, respectively, of the serrawettins with the silane linker. In the case of surfactin and bacillomycin, these lipopeptides have hydroxyl and carboxyl reactive groups on each structure. Instead of coupling the carboxylic acid group to N-hydroxysuccinimide to promote reaction with the terminal amine groups of the APTES, the hydroxyl groups could be subjected to oxidative reactions such as those described for the serrawettins.

The modified biosurfactant crude extracts (SB24, ST34, SB12, P1 and NP1) were then immobilized on the surfaces of the HDPE, PVC and stainless steel by carrying out the following steps:

### 3.2. Oxidation of Surfaces

The uncoated materials are typically unreactive. Wet chemistry treatment with a piranha etch solution was accordingly undertaken to provide hydroxyl groups on their surfaces. The schematic synthetic pathway shown in Scheme 3, below, illustrates the treatment with the piranha solution and subsequent steps.

All surfaces were washed with acetone and sterile Milli-Q water and dried. The coating was conducted in duplicate. The HDPE and PVC materials were immersed in piranha solution (4 mL in a test tube), consisting of 50% hydrogen peroxide and 50% concentrated sulphuric acid, for 30 min at room temperature. The stainless steel was immersed in piranha solution (4 mL in a test tube), consisting of 20% hydrogen peroxide and 80% concentrated sulphuric acid, for 1 hr at 100° C. After treatment, the materials were rinsed in Milli-Q water and ethanol, then dried under nitrogen.

There are other etching-based technologies for the functionalisation of surfaces. For example, oxygen plasma treatment can be used to oxidise polymeric surfaces; and electrochemical methods can be used to oxidise steel. These methods can have disadvantages, however. For example, plasma treatment may produce surface hydroxyl groups that are not stable for prolonged periods of time. This may reduce the likelihood of the hydroxyl groups coupling effectively with APTES linkers used for the biosurfactant bonding step.

3.3. Functionalisation of Surface with Silane Linker

In this step, the surface of the material was functionalised to promote reactivity with the modified biosurfactant. This step is illustrated in Scheme 3 below.

Advantageously, the silane linker employed for functionalising the surface of the material may be 3-triethoxysilylpropan-1-amine (APTES). Other silane linkers may optionally be used, such as those having thiol or carboxyl functionalities.

The piranha-treated materials were salinized in 3% APTES in dry ethanol (4 mL in a test tube) for 48 hrs. The APTES treated materials were washed in Milli-Q water and ethanol, soaked in ethanol for 10 min and sonicated for 10 min. The soaked surfaces were rinsed in ethanol to remove the non-attached APTES and were dried under nitrogen and placed in a desiccator for 10 min to stabilise the APTES monolayer.

3.4. Reaction of Biosurfactant with Functionalised Surface

In this step the modified biosurfactants were reacted with the surface of the APTES-functionalised material. This step is illustrated in Scheme 3 below. In this way the biosurfactant was immobilized on the surface of the material by covalent bonding with the APTES. The biosurfactant may be said to have become bioconjugated with the material.

The APTES-coated surfaces were immersed in 5 mg/mL of the modified biosurfactant compounds (in duplicate for each material) in dry ethanol, with continuous stirring at ambient temperature for 24 hrs. The coated surfaces were then washed in Milli-Q and ethanol and were dried under nitrogen. The coated and control (uncoated and APTES functionalised) materials were then stored at −20° C. until further analysis.

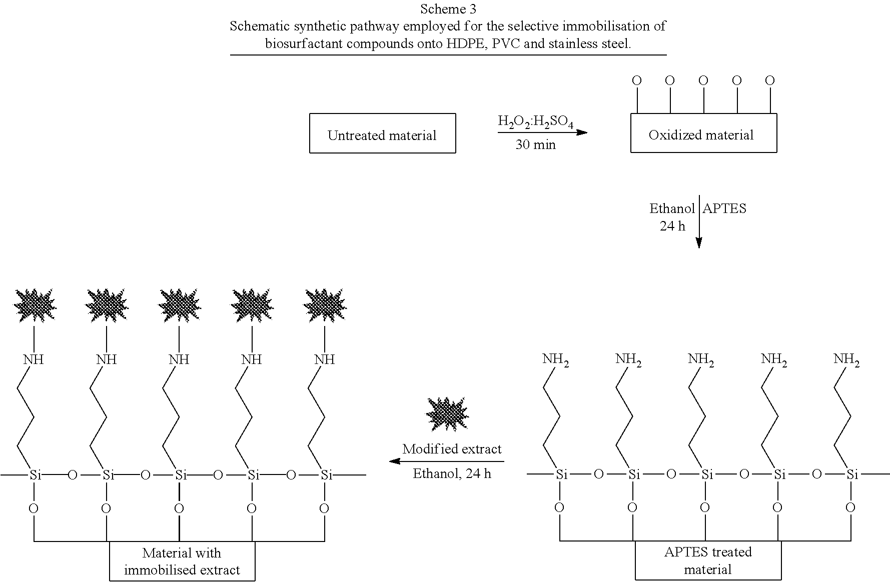

Scheme 3 is preferred as the pathway for covalently coupling the modified biosurfactant to the surface. Other coupling or bonding pathways may be feasible, such as crosslinking, grafting, oxidation, physical adsorption, and esterification. As previously noted, for example, the wells of a 96 microtiter plate coated with glycolipids from S. marcescens using simple adsorption methods may inhibit biofilms. However, such methods are distinct from the coating protocol disclosed herein, i.e. covalent immobilisation of biosurfactants. Furthermore, the types of biosurfactant used for the coating method disclosed herein are likewise distinct (e.g., lipopeptides produced by the S. marcescens P1 and NP1 strains).

The preferred pathway of Scheme 3 may thus have advantages over the abovementioned alternatives. These advantages may include the formation of stable covalent bonds between the APTES on the surface of the material and the modified biosurfactant compounds, which may result in a more stable modified surface over a longer period of time. In addition, the modified biosurfactant compounds may still display antimicrobial and antiadhesive properties.

### 4. SURFACE CHARACTERISATION

Surface characterisation methods were used to confirm the immobilisation of the biosurfactants on the surfaces of the materials. These included the use of water contact angle measurements, ATR-FTIR spectroscopy, scanning electron microscopy (SEM) and backscattered electron imaging-energy dispersive X-ray spectroscopy (BSE-EDX). The presence of various functional groups present on the surfaces of the materials (HDPE, PVC and stainless steel) was assessed.

Potential leaching of the immobilised biosurfactants was also determined using UPLC-ESI-MS.

### 4.1 Water Contact Angle Measurements

In order to confirm the successful immobilisation of the respective biosurfactants crude extracts onto the materials, the contact angle of water on the APTES-coated, biosurfactant-coated and uncoated materials (PVC, HDPE PE300 and stainless steel grade 304) was measured to assess the change in the surface wettability. A decrease in hydrophobicity was observed after the attachment of the biosurfactants onto the uncoated materials. The reduction in water contact angle provided an indication that the biosurfactant compounds had attached to the surface on the materials, due to the presence of the amine, hydroxyl and carboxyl groups (polar, hydrophilic functional groups) present in the biosurfactant structures.

### 4.2 Attenuated Total Reflectance Fourier Transform Infrared (ATR-FTIR) Spectroscopy

Surface characterisation was also carried out using an ATR-FTIR spectrometer. This confirmed the presence of functional groups and was used to compare the spectra of the uncoated, APTES-functionalised, and biosurfactant-coated PVC, HDPE and stainless steel samples. For HDPE and PVC functionalised with APTES after piranha treatment, detection of the functional groups NH₂, CH₂ and Si—O—Si on the surfaces of these materials confirmed that the APTES had successfully attached to the oxidised surfaces.

For the SB24 biosurfactant crude extract, the ATR-FTIR spectrum showed a broad peak at 3700-2800 cm⁻¹. This peak was an indication of the presence of the stretching —OH group which forms part of rhamnolipid congeners. In addition, persistence of an absorbance peak at approximately 3240 cm⁻¹ was an indication of the —NH₂ present in the modified SB24 biosurfactant crude extracts even after consumption of the terminal amine of the APTES-functionalised HDPE surface.

No major changes were observed for peaks at 2850 and 2950 cm⁻¹ in the APTES- and SB24-coated HDPE, which signified the presence of C—H bending and CH₂ groups, respectively, as expected for an HDPE material. A peak observed at 1660 cm⁻¹ confirmed the formation of amide bonds between the APTES and the modified SB24 biosurfactant extract. Another peak at approximately 1650 cm⁻¹ was assigned to —C═O groups existing in SB24 biosurfactant extract.

For the stainless steel samples, characteristic peaks from the APTES on the surface of the steel due to the methylene (CH₂) groups and primary amine were present. The ATR-FTIR analysis showed low absorbance peaks and was therefore inconclusive. This may possibly be explained by homogeneity of the stainless steel material. It will be appreciated by those skilled in the art that alternative analytical methods, such as Raman spectroscopy, may be used for the surface characterisation of homogeneous materials such as stainless steel (rather than ATR-FTIR). This may be of benefit if the homogeneity of the material makes it difficult to differentiate the coated from the uncoated material.

### 5. SURFACE MORPHOLOGY OF COATED MATERIALS

Scanning Electron Microscope (SEM) and Backscattered Electron Imaging-Energy Dispersive X-ray Spectroscopy (BSE-EDX) was used to visualise changes in surface morphology and elemental composition of the biosurfactant-coated HDPE, PVC and stainless steel samples in comparison to the uncoated controls.

A relatively smooth and uniform morphology was observed for the untreated HDPE sample. After surface modification with the biosurfactant crude extracts, a ‘granular-like’ topology was visible on the sample images coated with the SB24, SB12, P1 and NP1 biosurfactant crude extracts. However, the surface morphology of the HDPE displayed smoothness following immobilisation with ST34 biosurfactant crude extracts.

For the PVC samples, uniform narrow scratches were observed on the surface of the untreated samples. Following the coating of the PVC with the biosurfactant crude extracts, a ‘granular-like’ topology was again observed on the surface of the material coated with SB24 and NP1 biosurfactant crude extracts. Although the PVC coated with the P1 biosurfactant crude extract did not display a ‘granular-like’ appearance, a visible surface roughness was observed. In contrast, a smoother and uniform morphology was observed on the surface of PVC coated with ST34 and SB12.

For the stainless steel samples, a ‘tile-like’ morphology was apparent on the surface of the uncoated sample. Following biosurfactant immobilisation, an increase in surface smoothness was observed for the stainless steel coated with ST34 and P1 biosurfactant crude extracts. The SB12 immobilised surface on the other hand displayed a smoother texture, with a significant reduction in the ‘tile-like’ appearance. Although the ‘tile-like’ structure was still noticeable in the stainless steel samples immobilised with SB24 and NP1 biosurfactant crude extracts, a slight increase in the surface smoothness was observed with SB24, while the appearance of a hole-like surface with the NP1 biosurfactant crude extract was observed.

Overall, the observed changes in morphology and surface structure of the coated HDPE, PVC and stainless steel in comparison to the corresponding uncoated materials provided an indication that the material was successfully coated with the respective biosurfactant crude extracts.

### 6. STABILITY OF BIOSURFACTANT COMPOUNDS IMMOBILISED ON COATED MATERIALS

The stability of the biosurfactants immobilised on the materials was analysed. The coated material samples were each placed into a 250 mL flask containing 100 mL milliQ water. The flasks were incubated on an orbital shaker at ambient temperature and 15 mL of the suspension was collected after 24 hrs of incubation. The suspensions were lyophilised and dissolved in 15% acetonitrile to obtain a concentration of about 1.00 mg/mL. The samples were then subjected to UPLC-ESI-MS analysis, which revealed that no biosurfactant compounds were detected in the freeze-dried samples obtained after 24 hrs of incubation for all crude extracts tested (SB24, ST34, SB12, P1 and NP1). This provided an indication that no significant leaching of biosurfactant compounds had occurred, so the biosurfactants may be considered to be stable on HDPE, PVC and stainless steel for a minimum of 24 hrs in milliQ water.

### 7. ANTIFOULING ACTIVITY OF COATED MATERIALS

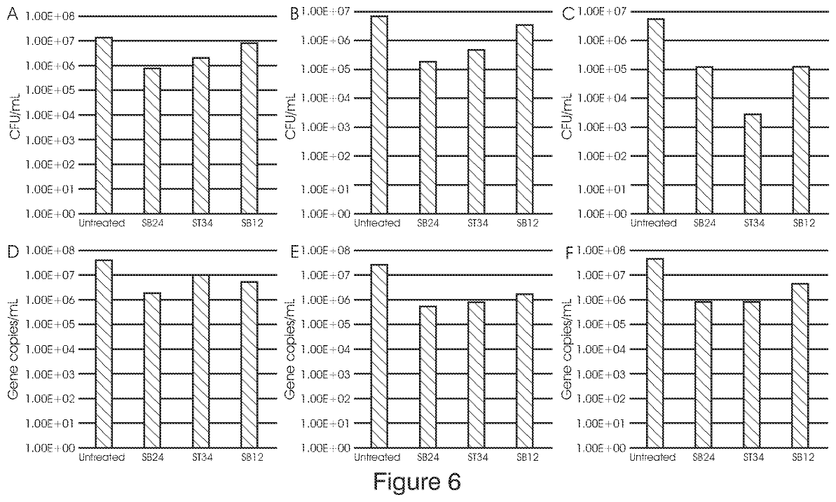

FIGS. 6-8: Plate Counts, EMA-qPCR Analysis and Confocal Laser Scanning Microscopy Following the confirmation of biosurfactant-immobilisation, the coated materials were subjected to laboratory-scale antifouling experiments to test the effectiveness of the coated materials to reduce the adhesion of selected test microorganisms that are commonly identified in food, water and clinical settings. These included Enterococcus faecalis (E. faecalis) S1, Pseudomonas aeruginosa (P. aeruginosa) S1 68 and Listeria monocytogenes (L. monocytogenes) C1.

The materials coated with SB24, ST34 and SB12 crude biosurfactant extracts were exposed to L. monocytogenes C1, while the materials coated with P1 and NP1 crude biosurfactant extracts were exposed to E. faecalis S1 or P. aeruginosa S1 68. Uncoated materials were included as negative controls.

Standard culture-based methods and EMA-qPCR analysis were used to evaluate the potential of the biosurfactants (SB24, ST34, SB12, P1 and NP1) immobilised onto HDPE, PVC and stainless steel to inhibit biofilm formation. Thus, the number of cells capable of adhering to the surface of the coated and uncoated materials were quantified.

Seed cultures of L. monocytogenes C1, E. faecalis S1 and P. aeruginosa S1 68 were prepared in 5 mL of Trypticase Soy Broth with 0.6% Yeast Extract (TSBYE_{0.6%}, Merck) and were incubated for 18-24 hrs at 37° C. The seed cultures were diluted to a final concentration of 10⁷ to 10⁹ CFU/mL using TSBYE_{0.6%}, which corresponds to an OD625 of 0.08 to 0.1. Subsequently, the coated materials were placed into test tubes containing 5 mL of the respective diluted seed culture, which was incubated at 37° C. for 18-24 hrs at 120 rpm. The analysis was conducted in duplicate. Uncoated surfaces were placed into 5 mL of sterile TSBYE_{0.6%} and were included as negative controls, while uncoated materials placed in a test tube containing 5 mL of each diluted seed culture of the respective microorganisms served as positive controls. After 24 hrs, the materials were removed from the test tubes and were rinsed with sterile saline (0.85%) to remove non-adherent microbial cells. The materials were then transferred to 3 mL sterile saline (0.85%) in a test tube and were sonicated for 5 min to recover the microbial cells that were able to attach to the surface of the coated and control materials. The resulting cell suspension was centrifuged at 10 000 rpm for 10 min to concentrate the microbial cells and was resuspended in 1 mL of saline (0.85%). The solution was serially diluted from 10⁰ to 10⁶ and 100 μL was spread plated onto TSAYE_{0.6%} plates. Plating was conducted in triplicate. The plates were then incubated at 37° C. for 24 hrs and the CFU/mL was determined.

EMA-qPCR was performed to quantify intact cells capable of adhering to the coated and control materials.

Confocal laser scanning microscopy was used to visually confirm the reduction in adhesion of L. monocytogenes C1, E. faecalis S1 and P. aeruginosa S1 68 cells onto the biosurfactant-immobilised materials. In order to visually confirm the reduction in microbial adhesion of L. monocytogenes C1, E. faecalis S1 and P. aeruginosa S1 68, each of the biosurfactant coated and control materials were stained with 3.35 μM SYTO-9 and 20 μM propidium iodide. Following staining, the uncoated and coated materials were examined for the viability of microbial cells using the confocal laser scanning microscope. Images were processed with respect to quality (live/dead ratio). All samples were sequentially scanned, frame-by-frame, first at 488 nm and then at 561 nm.

The results obtained for the uncoated and coated materials exposed to L. monocytogenes C1 will be discussed first, followed by the results obtained for the uncoated and coated materials exposed to the E. faecalis S1 strain, and then P. aeruginosa S1 68.

7.1. Biosurfactant Crude Extracts Obtained from Pseudomonas and Bacillus Species (FIG. 6)

FIG. 6 illustrates colony forming units and gene copies/mL of the L. monocytogenes C1 that were attached to uncoated and biosurfactant (SB24, ST34 and SB12) coated materials. The materials were analysed using plate counts (culture-based analysis) and EMA-qPCR analysis. The plate counts of L. monocytogenes C1 are presented in FIGS. 6A (HDPE), 6B (PVC), and 6C (stainless steel). The EMA-qPCR analyses of L. monocytogenes C1 are presented in FIGS. 6D (HDPE), 6E (PVC), and 6F (stainless steel).

Referring to FIG. 6, the ability of the SB24, ST34 and SB12 biosurfactant crude extracts coated onto HDPE to inhibit adhesion of L. monocytogenes C1 is presented in FIG. 6A. For the uncoated HDPE, an average of 1.48×10⁷ CFU/mL L. monocytogenes C1 cells was enumerated in the biofilm suspension using culture-based analysis. In comparison, for the SB24, ST34 and SB12 biosurfactant crude extracts coated onto HDPE, culture based analysis of the L. monocytogenes C1 cells in the biofilm suspension indicated that 7.87×10⁵ (94.67% inhibition), 2.25×10⁶ (84.72% inhibition) and 8.55×10⁶ (42.03% inhibition) CFU/mL were recorded, respectively. For the EMA-qPCR analysis, a qPCR efficiency of 2.04 (102%) was obtained, with a linear regression coefficient (R²) value of 1.00 recorded for the standard curve. Using the standard curve, viable L. monocytogenes C1 gene copy numbers were quantified in the biofilm suspension obtained for the uncoated and corresponding biosurfactant crude extract coated HDPE and are presented as prfA gene copies per mL (FIG. 6D). For the uncoated HDPE, an average of 3.82×10⁷ gene copies/mL was enumerated in the biofilm suspension using EMA-qPCR analysis (FIG. 6D). For the SB24, ST34 and SB12 biosurfactant extracts coated onto HDPE, EMA-qPCR analysis of the L. monocytogenes C1 cells in the biofilm suspension indicated that 1.82×10⁶ (95.22% inhibition), 9.94×10⁶ (73.97% inhibition) and 4.90×10⁶ (87.15% inhibition) gene copies/mL were recorded, respectively.

The ability of the SB24, ST34 and SB12 biosurfactant crude extracts coated onto PVC to inhibit adhesion of L. monocytogenes C1 is presented in FIG. 6B. For the uncoated PVC, an average of 7.26×10⁶ CFU/mL L. monocytogenes C1 cells was enumerated in the biofilm suspension using culture-based analysis (FIG. 6B).

For SB24, ST34 and SB12 coated onto PVC, culture based analysis of the L. monocytogenes C1 cells in the biofilm suspension indicated that 1.82×10⁵ (97.49% inhibition), 4.64×10⁵ (93.62% inhibition) and 3.54×10⁶ (51.23% inhibition) CFU/mL were recorded, respectively. For the EMA-qPCR (FIG. 6E), a qPCR efficiency of 2.04 (102%) was obtained, with a linear regression coefficient (R²) value of 1.00 recorded for the standard curve. Using the standard curve, viable L. monocytogenes C1 gene copy numbers were quantified in the biofilm suspension obtained for the uncoated and corresponding biosurfactant crude extract coated PVC and are presented as prfA gene copies per mL (FIG. 6E). For the uncoated PVC, an average of 2.18×10⁷ gene copies/mL L. monocytogenes C1 cells was enumerated in the biofilm suspension using EMA-qPCR analysis (FIG. 6E). For SB24, ST34 and SB12 coated onto PVC, EMA-qPCR analysis of the L. monocytogenes C1 cells in the biofilm suspension indicated that 4.84×10⁵ (97.78% inhibition), 8.53×10⁵ (96.09% inhibition) and 1.40×10⁶ (93.58% inhibition) gene copies/mL were recorded, respectively.

The ability of the SB24 biosurfactant crude extract coated onto stainless steel to inhibit adhesion of L. monocytogenes C1 is presented in FIG. 6C. For the uncoated stainless steel, an average of 5.61×10⁶ CFU/mL L. monocytogenes C1 cells was enumerated in the biofilm suspension using culture-based analysis (FIG. 6C). For the SB24, ST34 and SB12 biosurfactant crude extracts coated onto stainless steel, culture based analysis of the L. monocytogenes C1 cells in the biofilm suspension indicated that 1.19×10⁵ (97.87% inhibition), 2.46×10³ (99.96% inhibition) and 1.25×10⁵ (97.77% inhibition) CFU/mL were recorded, respectively. For the EMA-qPCR (FIG. 6F), a qPCR efficiency of 2.04 (102%) was obtained, with a linear regression coefficient (R²) value of 1.00 recorded for the standard curve. Using the standard curve, viable L. monocytogenes C1 gene copy numbers were quantified in the biofilm suspension obtained for the uncoated and corresponding biosurfactant crude extract coated stainless steel and are presented as prfA gene copies per mL (FIG. 6F). For the uncoated stainless steel, an average of 4.11×10⁷ gene copies/mL L. monocytogenes C1 cells was enumerated in the biofilm suspension using EMA-qPCR analysis (FIG. 6F). For the SB24, ST34 and SB12 biosurfactant extracts coated onto stainless steel, EMA-qPCR analysis of the L. monocytogenes C1 cells in the biofilm suspension indicated that 7.60×10⁵ (98.15% inhibition), 8.03×10⁵ (98.05% inhibition) and 3.84×10⁶ (90.66% inhibition) gene copies/mL were recorded, respectively.

Overall, in comparison to the L. monocytogenes C1 cells attached to the uncoated HDPE, PVC and stainless steel, a decrease in the L. monocytogenes C1 colony forming units and the prfA gene copy numbers was observed in the bacterial suspension obtained from the SB24, ST34 and SB12 crude extracts immobilised onto the respective materials. Based on the results of the plate counts and EMA-qPCR analysis, the SB24 coated HDPE and PVC displayed the highest anti-adhesion potential of the L. monocytogenes C1. However, based on the results of the plate counts and EMA-qPCR analysis, the highest anti-adhesion reduction of 99.96% and 98.05% were recorded for the ST34 and SB24 coated stainless steel, respectively.

### 7.1.1. Confocal Laser Scanning Microscopy

The ability of SB24, ST34 and SB12 biosurfactant extracts coated onto HDPE to inhibit L. monocytogenes C1 biofilm formation was also visualised using the LIVE/DEAD staining assay coupled to confocal laser scanning microscopy. After 20 hrs of exposure, L. monocytogenes C1 cells were able to colonise and form a biofilm on the uncoated HDPE surface. A significant reduction in viable biofilm cells was then observed on the surface of the HDPE coated with SB24, ST34 and SB12, while a corresponding increase in number of dead cells was apparent on the surface of the coated HDPE. Thus, the plate counts, EMA-qPCR and CLSM analysis indicate that coating HDPE with SB24, ST34 and SB12 biosurfactant extracts resulted in a reduction of L. monocytogenes C1 biofilm formation.

The ability of SB24, ST34 and SB12 biosurfactant extracts coated onto PVC to inhibit L. monocytogenes C1 biofilm formation was also visualised using the LIVE/DEAD staining assay coupled to confocal laser scanning microscopy. After 20 hrs of exposure, L. monocytogenes C1 cells were able to colonise and form a biofilm on the uncoated PVC surface. A significant reduction in viable L. monocytogenes C1 biofilm cells was then observed on the surface of the PVC coated with SB24, ST34 and SB12. Thus, the plate counts, EMA-qPCR and CLSM analysis indicate that coating PVC with SB24, ST34 and SB12 biosurfactant extracts resulted in a reduction of L. monocytogenes C1 biofilm formation.

Additionally, the ability of SB24, ST34 and SB12 biosurfactant extracts coated onto stainless steel to inhibit L. monocytogenes C1 biofilm formation was visualised using the LIVE/DEAD staining assay coupled to confocal laser scanning microscopy. After 20 hrs of exposure, L. monocytogenes C1 cells were able to colonise and form a biofilm on the uncoated stainless steel surface. A significant reduction in viable L. monocytogenes C1 biofilm cells was then observed on the surface of the stainless steel coated with SB24, ST34 and SB12. Thus, the plate counts, EMA-qPCR and CLSM analysis indicate that coating stainless steel with SB24, ST34 and SB12 biosurfactant extracts resulted in a reduction of L. monocytogenes C1 biofilm formation.

The confocal laser scanning microscopy images of the coated and uncoated materials (HDPE, PVC and stainless steel) showed that there was reduction in the bacterial cells attaching to the surfaces of the coated materials, which was attributed to the biosurfactant compounds immobilised onto the surfaces of these materials.

Without commitment to the veracity thereof, it is hypothesised that the immobilised biosurfactant compounds reduced attachment of the L. monocytogenes C1 on the HDPE, PVC and stainless steel by modification of the hydrophobicity of the coated materials and by increasing repulsive forces on their surfaces. The physicochemical properties of surfactin, bacillomycin and rhamnolipids may also reduce van der Waals forces that might otherwise reduce hydrophobic interactions between coated surfaces and bacterial cells.

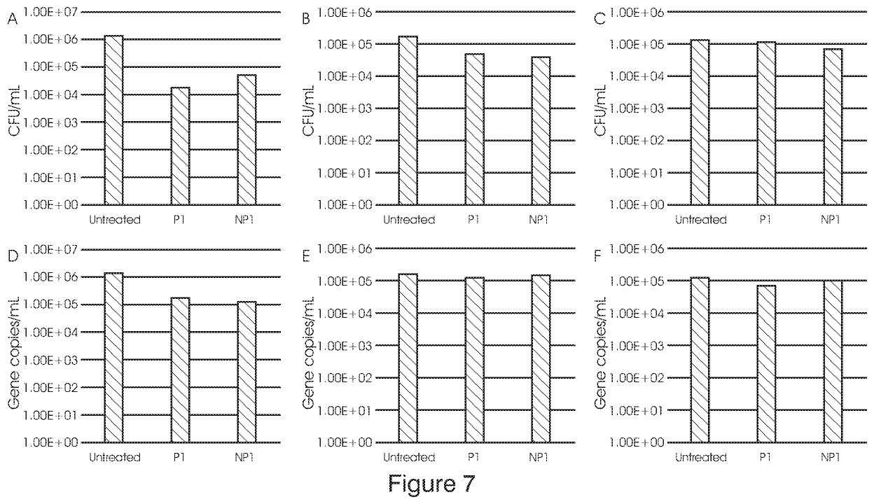

7.2. Biosurfactant Crude Extracts Obtained from S. marcescens Strains (FIG. 7)

Standard culture-based methods and EMA-qPCR analysis were also used to evaluate the antifouling properties of the P1 and NP1 biosurfactant crude extracts immobilised onto HDPE, PVC and stainless steel against E. faecalis S1.

FIG. 7 illustrates colony forming units and gene copies/mL of the E. faecalis S1 that were attached to uncoated and biosurfactant (P1 and NP1) immobilised materials.

The materials were analysed using plate counts (culture-based analysis) and EMA-qPCR analysis. The plate counts of E. faecalis S1 are presented in FIGS. 7A (HDPE), 7B (PVC), and 7C (stainless steel). The EMA-qPCR analyses of E. faecalis S1 are presented in FIGS. 7D (HDPE), 7E (PVC), and 7F (stainless steel).

For the uncoated HDPE, an average of 1.46×10⁶ CFU/mL E. faecalis S1 cells was enumerated in the biofilm suspension using culture-based analysis (FIG. 7A). For the P1 and NP1 biosurfactant crude extracts immobilised onto HDPE, culture-based analysis of the E. faecalis S1 cells in the biofilm suspension indicated that 1.86×10⁴ (98.73% inhibition) and 5.20×10⁴ (96.44% inhibition) CFU/mL were recorded, respectively.

In addition to the culturing analysis, the potential of the P1 and NP1 biosurfactant extracts immobilised onto HDPE to inhibit biofilm formation by E. faecalis S1 was evaluated using EMA-qPCR (FIG. 7D). A qPCR efficiency of 2.03 (102%) was obtained, with a linear regression coefficient (R²) value of 1.00 recorded for the standard curve. Using the standard curve, viable E. faecalis S1 gene copy numbers were quantified in the biofilm suspension obtained for the uncoated and corresponding biosurfactant crude extract immobilised HDPE and are presented as 23S rRNA gene copies per mL (FIG. 7D). For the uncoated HDPE, an average of 1.25×10⁶ gene copies/mL was enumerated in the biofilm suspension using EMA-qPCR analysis (FIG. 7D). For the P1 and NP1 biosurfactant extracts immobilised onto HDPE, EMA-qPCR analysis of the E. faecalis S1 cells in the biofilm suspension indicated that 1.59×10⁵ (87.23% inhibition) and 1.12×10⁵ (91.02% inhibition) gene copies/mL were recorded, respectively.

Referring to FIG. 7, the ability of the P1 and NP1 biosurfactant crude extract coated onto PVC to inhibit adhesion of E. faecalis S1 is presented in FIG. 7B. For the uncoated PVC, an average of 1.79×10⁵ CFU/mL E. faecalis S1 cells was enumerated in the biofilm suspension using culture-based analysis (FIG. 7B). For the P1 and NP1 biosurfactant crude extracts immobilised onto PVC, culture-based analysis of the E. faecalis S1 cells in the biofilm suspension indicated that 4.88×10⁴ (72.74% inhibition) and 4.15×10⁴ (76.82% inhibition) CFU/mL were recorded, respectively. For the EMA-qPCR analysis (FIG. 7E), a qPCR efficiency of 2.03 (102%) was obtained, with a linear regression coefficient (R²) value of 1.00 recorded for the standard curve. Using the standard curve, viable E. faecalis S1 gene copy numbers were quantified in the biofilm suspension obtained for the uncoated and corresponding biosurfactant crude extract immobilised PVC and are presented as 23S rRNA gene copies per mL (FIG. 7E).

For the uncoated PVC, an average of 1.54×10⁵ gene copies/mL was enumerated in the biofilm suspension using EMA-qPCR analysis (FIG. 7E). For the P1 and NP1 biosurfactant extracts immobilised onto PVC, EMA-qPCR analysis of the E. faecalis S1 cells in the biofilm suspension indicated that 1.13×10⁵ (26.59% inhibition) and 1.41×10⁵ (8.75% inhibition) gene copies/mL were recorded, respectively.

The ability of the P1 and NP1 biosurfactant crude extract coated onto stainless steel to inhibit adhesion of E. faecalis S1 is presented in FIG. 7C. For the uncoated stainless steel, an average of 1.40×10⁵ CFU/mL E. faecalis S1 cells was enumerated in the biofilm suspension using culture-based analysis (FIG. 7C). For the P1 and NP1 biosurfactant crude extracts immobilised onto stainless steel, culture-based analysis of the E. faecalis S1 cells in the biofilm suspension indicated that 1.14×10⁵ (18.43% inhibition) and 7.19×10⁴ (37.16% inhibition) CFU/mL were recorded, respectively. For the EMA-qPCR analysis (FIG. 7F), a qPCR efficiency of 2.03 (102%) was obtained, with a linear regression coefficient (R²) value of 1.00 recorded for the standard curve. Using the standard curve, viable E. faecalis S1 gene copy numbers were quantified in the biofilm suspension obtained for the uncoated and corresponding biosurfactant crude extract immobilised stainless steel and are presented as 23S rRNA gene copies per mL (FIG. 7F). For the uncoated stainless steel, an average of 1.20×10⁵ gene copies/mL E. faecalis S1 cells was enumerated in the biofilm suspension using EMA-qPCR analysis (FIG. 7F). For the P1 and NP1 biosurfactant extracts immobilised onto stainless steel, EMA-qPCR analysis of the E. faecalis S1 cells in the biofilm suspension indicated that 6.82×10⁴ (43.17% inhibition) and 9.72×10⁴ (19.09% inhibition) gene copies/mL were recorded, respectively.

### 7.2.1. Confocal Laser Scanning Microscopy

The ability of P1 and NP1 biosurfactant extracts immobilised onto HDPE to inhibit E. faecalis S1 biofilm formation was visualised using the LIVE/DEAD staining assay coupled to confocal laser scanning microscopy. After 20 hrs of exposure, E. faecalis S1 cells were able to colonise and form a biofilm on the uncoated HDPE surface. A significant reduction in viable biofilm cells was then observed on the surface of the HDPE immobilised with P1 and NP1, while an increase in number of dead cells was also apparent on the surface of the immobilised HDPE. Thus, the plate counts, EMA-qPCR and CLSM analysis indicate that coating HDPE with P1 and NP1 biosurfactant extracts resulted in a reduction of E. faecalis S1 biofilm formation.

The ability of P1 and NP1 biosurfactant extracts immobilised onto PVC to inhibit E. faecalis S1 biofilm formation was also visualised using the LIVE/DEAD staining assay coupled to confocal laser scanning microscopy. After 20 hrs of exposure, E. faecalis S1 cells were able to colonise and form a biofilm on the uncoated PVC surface. A significant reduction in viable biofilm cells was then observed on the surface of the PVC immobilised with P1 and NP1, while an increase in number of dead cells was also apparent on the surface of the immobilised PVC. Thus, the plate counts and CLSM analysis indicate that coating PVC with P1 and NP1 biosurfactant extracts resulted in a reduction of E. faecalis S1 biofilm formation.

The ability of P1 and NP1 biosurfactant extracts immobilised onto stainless steel to inhibit E. faecalis S1 biofilm formation was also visualised using the LIVE/DEAD staining assay coupled to confocal laser scanning microscopy. After 20 hrs of exposure, E. faecalis S1 cells were able to colonise and form a biofilm on the uncoated stainless steel surface. Although a visual reduction in viable biofilm cells was observed on the surface of the stainless steel following immobilisation with P1 and NP1, live cells were still apparent on the surface of the immobilised stainless steel.

For the HDPE coated with P1 and NP1, the highest reduction in E. faecalis S1 was observed for P1 with a 98.73% reduction in CFU; however, the gene copies obtained for P1 and NP1 were comparable.

Similarly, the highest reduction in E. faecalis S1 for the P1 and NP1 extracts immobilised onto PVC was observed for NP1 with a 76.82% reduction CFU, while the EMA-qPCR analysis revealed that no significant reduction in E. faecalis S1 gene copies in comparison to the uncoated control. For the stainless steel coated with P1 and NP1, no significant reductions in E. faecalis S1 CFU or gene copies were observed.

In summary, although the immobilised biosurfactants did not fully inhibit biofilm formation of E. faecalis S1 after 24 hrs, the HDPE coated materials showed significantly reduced microbial attachment in comparison to the uncoated control. Confocal laser scanning microscopy further confirmed the reduction in E. faecalis S1 cells attaching to the surfaces of the coated HDPE and PVC materials in comparison to the respective uncoated materials, while comparable results were obtained for stainless steel surfaces.

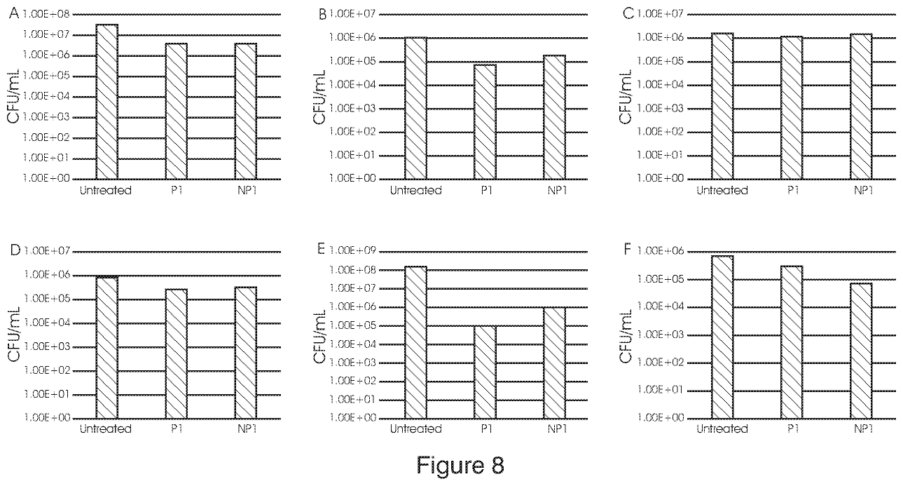

7.3. Biosurfactant Crude Extracts Obtained from S. marcescens Strains (FIG. 8)

Standard culture-based methods and EMA-qPCR analysis were also used to evaluate the antifouling properties of the P1 and NP1 biosurfactant crude extracts immobilised onto HDPE, PVC and stainless steel against P. aeruginosa S1 68.

FIG. 8 illustrates colony forming units and gene copies/mL of the P. aeruginosa S1 68 that were attached to uncoated and biosurfactant (P1 and NP1) immobilised materials.

The materials were analysed using plate counts (culture-based analysis) and EMA-qPCR analysis. The plate counts of P. aeruginosa S1 68 are presented in FIGS. 8A (HDPE), 8B (PVC), and 8C (stainless steel). The EMA-qPCR analyses of E. faecalis S1 are presented in FIGS. 8D (HDPE), 8E (PVC), and 8F (stainless steel).

For the uncoated HDPE, an average of 3.86×10⁷ CFU/mL P. aeruginosa S1 68 cells was enumerated in the biofilm suspension using culture-based analysis (FIG. 8A). For the P1 and NP1 biosurfactant crude extracts immobilised onto HDPE, culture-based analysis of the P. aeruginosa S1 68 cells in the biofilm suspension indicated that 4.90×10⁶ (87.31% inhibition) and 4.78×10⁶ CFU/mL (87.61% inhibition) CFU/mL were recorded, respectively.

In addition to the culturing analysis, the potential of the P1 and NP1 biosurfactant extracts immobilised onto HDPE to inhibit biofilm formation by P. aeruginosa S1 68 was evaluated using EMA-qPCR (FIG. 8D). A qPCR efficiency of 1.96 (98%) was obtained, with a linear regression coefficient (R²) value of 1.00 recorded for the standard curve. Using the standard curve, viable P. aeruginosa S1 68 gene copy numbers were quantified in the biofilm suspension obtained for the uncoated and corresponding biosurfactant crude extract immobilised HDPE and are presented as oprl gene copies per mL (FIG. 8D). For the uncoated HDPE, an average of 1.01×10⁶ gene copies/mL was enumerated in the biofilm suspension using EMA-qPCR analysis (FIG. 8D). For the P1 and NP1 biosurfactant extracts immobilised onto HDPE, EMA-qPCR analysis of the P. aeruginosa S1 68 cells in the biofilm suspension indicated that 2.83×10⁵ (72.12% inhibition) and 3.62×10⁵ gene copies/mL (64.33% inhibition) gene copies/mL were recorded, respectively.

Referring to FIG. 8, the ability of the P1 and NP1 biosurfactant crude extract coated onto PVC to inhibit adhesion of P. aeruginosa S1 68 is presented in FIG. 8B. For the uncoated PVC, an average of 1.09×10⁶ CFU/mL P. aeruginosa S1 68 cells was enumerated in the biofilm suspension using culture-based analysis (FIG. 8B). For the P1 and NP1 biosurfactant crude extracts immobilised onto PVC, culture-based analysis of the P. aeruginosa S1 68 cells in the biofilm suspension indicated that 7.88×10⁴ (92.77% inhibition) and 2.09×10⁵ CFU/mL (80.87% inhibition) CFU/mL were recorded, respectively. For the EMA-qPCR analysis (FIG. 8E), a qPCR efficiency of 1.96 (98%) was obtained, with a linear regression coefficient (R²) value of 1.00 recorded for the standard curve. Using the standard curve, viable P. aeruginosa S1 68 gene copy numbers were quantified in the biofilm suspension obtained for the uncoated and corresponding biosurfactant crude extract immobilised PVC and are presented as oprl gene copies per mL (FIG. 8E).

For the uncoated PVC, an average of 1.71×10⁸ gene copies/mL was enumerated in the biofilm suspension using EMA-qPCR analysis (FIG. 8E). For the P1 and NP1 biosurfactant extracts immobilised onto PVC, EMA-qPCR analysis of the P. aeruginosa S1 68 cells in the biofilm suspension indicated that 9.59×10⁴ (99.94% inhibition) and 9.84×10⁵ gene copies/mL (99.43% inhibition) gene copies/mL were recorded, respectively.

The ability of the P1 and NP1 biosurfactant crude extract coated onto stainless steel to inhibit adhesion of P. aeruginosa S1 68 is presented in FIG. 8C. For the uncoated stainless steel, an average of 1.83×10⁶ CFU/mL P. aeruginosa S1 68 cells was enumerated in the biofilm suspension using culture-based analysis (FIG. 8C). For the P1 and NP1 biosurfactant crude extracts immobilised onto stainless steel, culture-based analysis of the P. aeruginosa S1 68 cells in the biofilm suspension indicated that 1.29×10⁶ (29.24% inhibition) and 1.65×10⁶ CFU/mL (9.95% inhibition) CFU/mL were recorded, respectively. For the EMA-qPCR analysis (FIG. 8F), a qPCR efficiency of 1.96 (98%) was obtained, with a linear regression coefficient (R²) value of 1.00 recorded for the standard curve. Using the standard curve, viable P. aeruginosa S1 68 gene copy numbers were quantified in the biofilm suspension obtained for the uncoated and corresponding biosurfactant crude extract immobilised stainless steel and are presented as oprl gene copies per mL (FIG. 8F). For the uncoated stainless steel, an average of 7.48×10⁵ gene copies/mL P. aeruginosa S1 68 cells was enumerated in the biofilm suspension using EMA-qPCR analysis (FIG. 8F). For the P1 and NP1 biosurfactant extracts immobilised onto stainless steel, EMA-qPCR analysis of the P. aeruginosa S1 68 cells in the biofilm suspension indicated that 3.14×10⁵ (58.02% inhibition) and 7.61×10⁴ gene copies/mL (89.83% inhibition) gene copies/mL were recorded, respectively.

### 7.3.1. Confocal Laser Scanning Microscopy

The ability of P1 and NP1 biosurfactant extracts immobilised onto HDPE to inhibit P. aeruginosa S1 68 biofilm formation was visualised using the LIVE/DEAD staining assay coupled to confocal laser scanning microscopy. After 20 hrs of exposure, P. aeruginosa S1 68 cells were able to colonise and form a biofilm on the uncoated HDPE surface. A reduction in viable biofilm cells was then observed on the surface of the HDPE immobilised with P1 and NP1, while an increase in number of dead cells was also apparent on the surface of the immobilised HDPE. Thus, the plate counts, EMA-qPCR and CLSM analysis indicate that coating HDPE with P1 and NP1 biosurfactant extracts resulted in a reduction of P. aeruginosa S1 68 biofilm formation.

The ability of P1 and NP1 biosurfactant extracts immobilised onto PVC to inhibit P. aeruginosa S1 68 biofilm formation was also visualised using the LIVE/DEAD staining assay coupled to confocal laser scanning microscopy. After 20 hrs of exposure, P. aeruginosa S1 68 cells were able to colonise and form a biofilm on the uncoated PVC surface. A significant reduction in viable biofilm cells was then observed on the surface of the PVC immobilised with P1 and NP1, while an increase in number of dead cells was also apparent on the surface of the immobilised PVC. Thus, the plate counts and CLSM analysis indicate that coating PVC with P1 and NP1 biosurfactant extracts resulted in a reduction of P. aeruginosa S1 68 biofilm formation.

The ability of P1 and NP1 biosurfactant extracts immobilised onto stainless steel to inhibit P. aeruginosa S1 68 biofilm formation was also visualised using the LIVE/DEAD staining assay coupled to confocal laser scanning microscopy. After 20 hrs of exposure, P. aeruginosa S1 68 cells were able to colonise and form a biofilm on the uncoated stainless steel surface. A clear reduction in viable biofilm cells was not observed on the stainless steel coated with NP1 biosurfactant extract, while minor reductions in viable biofilm cells were observed for the stainless steel coated with P1 biosurfactant extract.

For the HDPE coated with P1 and NP1, the reduction of P. aeruginosa S1 68 CFU was comparable for P1 and NP1 at 87.3% and 87.6% respectively, while the highest reduction of P. aeruginosa S1 68 gene copies was observed for P1 at 72.1% reduction in gene copies compared to the control.

For the PVC coated with P1 and NP1, the highest reduction in P. aeruginosa S1 68 for the P1 and NP1 extracts immobilised onto PVC was observed for P1 with a 92.8% reduction based on CFU; however, the gene copies obtained for P1 and NP1 were comparable.

For the stainless steel coated with P1 and NP1, no significant reductions in P. aeruginosa S1 68 CFU were observed, while minor reductions in P. aeruginosa S1 68 gene copies were observed.

In summary, although the coated biosurfactants did not fully inhibit biofilm formation of P. aeruginosa S1 68 after 24 hrs, the polymeric surfaces (HDPE and PVC) coated with P1 and NP1 biosurfactant extracts significantly reduced microbial attachment of P. aeruginosa S1 68 in comparison to the uncoated control materials. Confocal laser scanning microscopy further confirmed the reduction in P. aeruginosa S1 68 cells attaching to the surfaces of the coated HDPE and PVC materials in comparison to the respective uncoated materials, while minor reductions in viable biofilm cells were observed for stainless steel coated with P1.

### 8. CONCLUSIONS

In comparison to the uncoated HDPE and PVC controls, the culture based analysis and EMA-qPCR results indicated an overall decrease in the adhesion of L. monocytogenes C1 cells for all the biosurfactant crude extracts coated onto HDPE and PVC. However, the culture based analysis and EMA-qPCR results indicated that the SB24 immobilised HPDE and PVC had the highest decrease in the adhesion of L. monocytogenes C1, with 94.67% and 95.22% (SB24 immobilised HDPE) and 97.49% and 97.78% (SB24 immobilised PVC) inhibition, respectively.

For the SB24, ST34 and SB12 immobilised onto stainless steel, the culture based analysis and EMA-qPCR results indicated an overall decrease in the adhesion of L. monocytogenes C1 cells. However, the culture based analysis and EMA-qPCR results indicated that the highest decrease in the adhesion of L. monocytogenes C1 was obtained for ST34 immobilised onto stainless steel, with 94.67% and 95.22% inhibition, respectively.

The immobilised biosurfactant compounds thus appear to have inhibited attachment of L. monocytogenes C1 onto the HDPE, PVC and stainless steel.

For the HDPE coated with P1 and NP1, the highest reduction in E. faecalis S1 was observed for P1 with a 98.73% reduction in CFU; however, gene copies revealed that P1 and NP1 has similar reduction percentages for E. faecalis S1.

Similarly, the highest reduction in E. faecalis S1 for the P1 and NP1 extracts immobilised onto PVC was observed for NP1 with a 76.82% reduction in CFU, while the EMA-qPCR analysis revealed that no significant reduction in E. faecalis S1 gene copies were observed in comparison to the uncoated control.

For the stainless steel coated with P1 and NP1, no significant reductions of E. faecalis S1 were observed for CFU and gene copies.

Confocal laser scanning microscopy confirmed a reduction in E. faecalis S1 cells on the surface of the P1 and NP1 coated HDPE and PVC versus the uncoated controls, while comparable results were obtained for the coated stainless steel surfaces versus the uncoated controls.

For the HDPE coated with P1 and NP1, the reduction of P. aeruginosa S1 68 CFU was comparable for P1 and NP1 at 87.3 and 87.6% respectively, while the highest reduction of P. aeruginosa S1 68 gene copies was observed for P1 at 72.1% reduction in gene copies compared to the control.

For the PVC coated with P1 and NP1, the highest reduction in P. aeruginosa S1 68 for the P1 and NP1 extracts immobilised onto PVC was observed for P1 with a 92.8% reduction based on CFU; however, the gene copies obtained for P1 and NP1 were comparable.

For the stainless steel coated with P1 and NP1, no significant reductions in P. aeruginosa S1 68 CFU were observed, while minor reductions in P. aeruginosa S1 68 gene copies were observed.

Confocal laser scanning microscopy confirmed a reduction in P. aeruginosa S1 68 cells on the surface of the P1 and NP1 coated HDPE and PVC, and the P1 coated stainless steel versus the uncoated controls, while comparable results were obtained for the NP1 coated stainless steel surfaces versus the uncoated controls.

Overall, the following conclusions may be drawn:
Biosurfactant crude extracts produced by P. aeruginosa SB24 may effectively reduce the adhesion of L. monocytogenes cells onto HDPE and PVC.
Biosurfactant crude extracts produced by B. amyloliquefaciens ST34 may effectively reduce the adhesion of L. monocytogenes cells onto stainless steel.
Biosurfactant crude extracts produced by S. marcescens P1 and NP1 may effectively reduce the adhesion of E. faecalis S1 and P. aeruginosa onto HDPE.
Biosurfactant crude extracts produced by S. marcescens P1 and NP1 may effectively reduce the adhesion of P. aeruginosa onto PVC.

It will be appreciated by those skilled in the art that the species of biofilm-forming microorganisms that were targeted in the exemplary methods described above are representative only; and that biofilms of other species of microorganisms may also be expected to be inhibited, disrupted or dispersed by the same biosurfactants coated onto PVC, HDPE and stainless steel.

For example, and without commitment to the veracity thereof, HDPE, PVC and stainless steel materials that are coated with rhamnolipid according to the methods of the invention may have the potential to inhibit biofilms formed by Escherichia coli, Klebsiella pneumoniae, methicillin-resistant Staphylococcus aureus (MRSA) and Cryptococcus neoformans strains, based on antimicrobial and antiadhesive activities observed against these bacterial species.

By way of further example, and again without commitment to the veracity thereof, HDPE, PVC and stainless steel materials that are coated with the ST34 and SB12 extracts (composed of surfactin and bacillomycin analogues and homologues) may have the potential to inhibit biofilms of Legionella pneumophila, Legionella longbeachae, Staphylococcus equorum and methicillin-resistant Staphylococcus aureus (MRSA); and those coated with the P1 and NP1 extracts (composed of serrawettin W1 homologues and glucosamine derivative homologues) may have the potential to inhibit biofilm formation by Listeria monocytogenes, Acinetobacter baumannii, Cryptococcus neoformans and Candida albicans.

It will be appreciated by those skilled in the art that further processing of the materials disclosed herein may be undertaken. For example, the crude extracts of SB24, ST34, SB12, P1 and NP1 may be subjected to high performance liquid chromatography to obtain purer fractions of the respective biosurfactant compounds. The antiadhesive potential of the purified fractions may be investigated using the MBEC™ assay to determine those fractions exhibiting the highest antiadhesive potential. The described methods may then be used to immobilise those fractions onto HDPE, PVC and stainless steel for the inhibition of biofilm formation.

In addition to the three species of microorganisms specifically identified for use with the described coating methods, other microorganisms may also be suitable for producing the biosurfactants used. For example, biosurfactants like those immobilised in described coating methods may be derived from the following microorganisms:
Bacillus subtilis, Bacillus velezensis, Bacillus mojavensis and Bacillus amyloliquefaciens strains may produce analogues and homologues of surfactin biosurfactants;
Bacillus subtilis, Bacillus mojavensis and Bacillus amyloliquefaciens may produce bacillomycin L and bacillomycin D biosurfactants;
Pseudomonas aeruginosa, Pseudomonas putida, Burkholderia kururiensis, Burkholderia plantarii, Burkholderia thailandensis and Burkholderia glumae strains may produce rhamnolipid congeners; and
Serratia marcescens and Serratia surfactantfaciens may produce serrawettin W1 homologues and analogues.

The described methods and protocols may be employed for coating materials with biosurfactants produced by these additional species of microorganism, along with any other microorganism capable of producing the identified biosurfactants.

It will also be appreciated that other materials may be coated using the described methods and protocols. These materials may include polymers such as poly(dimethyl siloxane), polypropylene, polystyrene, acrylonitrile-butadienestyrene (ABS), silicone, glass, ceramics and various alternative grades of stainless steel. These materials may be suitable for the surface oxidisation step (e.g., the treatment with piranha solution) and for surface modification with the biosurfactant compounds.

### Possible Mechanisms of Antifouling Activity

Without commitment to the veracity thereof, the following mechanisms and modes of action may play a role in the observed antifouling activity of the coated materials. Firstly, changes in the hydrophobicity of the materials based on water contact angle measurements suggested that their surfaces may have been modified to be more hydrophilic. The biosurfactants may also be capable of modifying the physicochemical properties of the surfaces and of reducing the adhesion of microorganisms and hence the formation of biofilms.

A general mode of action has been proposed for lipopeptides and glycolipids. Lipopeptides such as serrawettins and surfactin are amphipathic in nature, meaning that they are composed of a peptide moiety (such as varying number and composition of amino acids) attached to a fatty acid moiety (such as one or more fatty acid chains of varying length. Lipopeptides may permit binding to the lipid (hydrophobic) and the phospholipid (hydrophilic) regions of bacterial cell membranes based on amphipathic interactions. In addition, the electrostatic charge of the hydrophilic moiety and the length of the lipid may contribute to the antimicrobial activity. Upon binding to the membrane, lipopeptides can accumulate on the surface of a microbial cell (bacteria and fungi) until a threshold concentration is reached, where after they permeate the membrane leading to its disintegration. This disintegration is induced by a detergent-like mechanism and may occur by the formation of pores in the cell membrane of microbial cells, thus increasing the influx of Ca²⁺ and H⁺ into the cells. The pore formation may cause an imbalance in transmembrane ion fluxes and cell death.

Glycolipids such as rhamnolipids have hydrophilic moieties made up of mono-, di-, tri- or tetra-saccharide carbohydrates. These are attached to different (chain length) hydrophobic moieties which form a lipid backbone. Glycolipids have structures and properties similar to those of detergents and may be able to intercalate into the membrane phospholipid bilayer of a microbial cell, thereby facilitating the permeability of the membrane and flow of metabolites out of the cell. The intercalation may alter the structure and function of the phospholipid bilayer through the interruption of the protein conformation. Thus, transport and energy generation may be disrupted and the process can be lethal to various Gram-positive bacteria. Rhamnolipids may also decrease the adhesiveness of cell-cell, cell-matrix, and cell-surface interactions, and induce central hollowing and biofilm detachment.

The described coating methods may have advantages over other methods of coating surfaces with biosurfactants. They may be suitable for reducing the adhesion of potentially pathogenic bacteria and in so doing inhibit biofilm formation. This may be advantageous as it may reduce microbial contamination and biofouling of a variety of things, including but not limited to water distribution equipment, medical devices and implants, and food processing plants and surfaces. This may, in turn, save costs which would otherwise need to be spent to remedy such contamination and biofouling. Illnesses caused by microbial contamination may also be avoided or mitigated.

Other methods of coating materials with antimicrobial compounds are known, such as physical absorption or adsorption, ion linkage, crosslinking and polymerisation, or encapsulation or incorporation of an antimicrobial substance into a material. There are several limitations to these methods, however, including loss of antifouling potency over time, potential toxicity or the development of antimicrobial resistance due to a low concentration of the compound being released. The coating and immobilization methods describe herein, by contrast, provide covalent linkages between the antimicrobial compound and the APTES-functionalised material. The covalent nature of the bonding may be expected to reduce leaching, enhance long-term stability and increase the duration of antimicrobial efficacy. The covalent coupling which is achieved by the disclosed method may provide a stronger bond to the functional groups than may be obtained by absorption, adsorption or ionic linking.

In regard to the modification of the serrawettin and glucosamine derivatives in the P1 and NP1 crude extracts (by substitution of the hydroxyl groups with chlorine), those skilled in the art will appreciate that alternative modification routes, while feasible, may not be as effective as chlorine substitution. Based on the structure of serrawettin W1 homologues, the main reactive groups are the primary hydroxyl groups, and glucosamine derivatives have primary hydroxyl groups and alkene moieties as reactive groups. To promote immobilization of the serrawettins and glucosamine derivatives on silanized surfaces resulting from functionalisation with APTES, an effective and strong covalent bond with the amine functionalities on the APTES is crucial. Reaction between primary amines and chlorine groups meets this criterion and may be more cost-effective than alternative modification strategies such as oxidation to an aldehyde or a carboxylic acid.

### INDUSTRIAL APPLICABILITY

The coated materials and articles of manufacture described herein may have antiadhesive, biofilm disrupting and antifouling activity.

The coated PVC, HDPE and stainless steel materials may be suitable for inhibiting and disrupting biofilms in various applications, including but not limited to:
the water industry (inter alia for water-storage tanks and water conveyance and distribution apparatus, pipes, taps, valves and the like; application of biosurfactants to the surfaces of such apparatus may be useful for biocontrol and antifouling)
the medical industry (inter alia for medical devices and implants, e.g., catheters, and for use in hospitals, clinical settings, and biomedical and biotechnology industries)
the food industry (inter alia for food vending equipment and food processing plants and equipment)
general industrial processes (inter alia for cooling systems where biofouling may occur)
the marine and shipping industries (inter alia for inhibition of biofouling on vessel hulls or the surfaces of other marine apparatus)

### Unity of Invention

The Bacillus, Pseudomonas and Serratia strains forming the subject matter of the disclosed methods and materials together constitute a unified subset or group of strains. The subset is unified by the broad-spectrum antimicrobial activity of the biosurfactants which they produce, being effective for reducing the adhesion of a wide range of microorganisms. The group of strains was selected by a multistep procedure. Firstly, a consortium of bacterial strains was subjected to screening tests to identify strains that were capable of biosurfactant production. The screening methods included oil spreading methods, emulsification index assays and surface tension reduction measurements. The biosurfactants produced by twelve strains identified by the screening were then extracted by solvent extraction methods and the extracts were subjected to antimicrobial testing against a wide range of Gram-positive and Gram-negative opportunistic and pathogenic bacteria, as well as fungal pathogens. Based on this testing, the disclosed strains of Bacillus, Pseudomonas and Serratia were selected. The three strains accordingly form part of a unified selection or subset based not only on the effectiveness of the biosurfactants they produce, but also on their manner of selection.

The materials which were coated by the method of the invention (PVC, HDPE and stainless steel) also form a unified set of materials. They are united as a group by their common use in applications requiring the inhibition of biofilm formation for industrial and economic reasons and for the mitigation of health risks. As illustrated above, they are commonly and widely utilised in the water distribution industry, the medical industry and the food industry, amongst others. All three materials are used as piping materials, for example. PVC is used for constructing water distribution pipes (for municipal and industrial applications), medical devices, single-use containers (containers used for blood components), tubing used in the medical industry (e.g., for blood transfusions, heart-lung bypasses and haemodialysis, as well as for catheters), flooring in hospitals, non-food packaging and food-covering sheets. HDPE is used to manufacture water pipes for domestic water supply and agricultural processes, water storage tanks, inner linings of interventional catheters, certain plastic based medical implants and food packaging. Stainless steel is used to manufacture storage tanks and tankers for water, food and beverage products, surgical instruments, surgical implants (such as bone reinforcements and replacements), industrial equipment used in water treatment plants, and food-processing and commercial kitchen equipment, amongst other uses. Various opportunistic and pathogenic bacteria and fungi are capable of forming biofilms on the surface of these three materials, which can result in significant health risks and financial losses.

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Throughout the specification and claims unless the context requires otherwise the word ‘comprise’ or variations such as ‘comprises’ or ‘comprising’ will be understood to imply the inclusion of a stated integer or group of integers but not the exclusion of any other integer or group of integer

1. A coating method for coating a surface of a substrate material with a biosurfactant, the method comprising modifying the biosurfactant to promote its reactivity with a silane linker; oxidising the surface of the substrate material; functionalising the surface of the substrate material with a silane linker; and reacting the modified biosurfactant with the functionalised surface, thereby covalently to bond the biosurfactant to the surface of the substrate material.

2. The coating method as claimed in claim 1, wherein the substrate material is selected from the group consisting of polymers and ferrous metals.

3. The coating method as claimed in claim 2, wherein the substrate material is selected from the group consisting of high-density polyethylene, polyvinyl chloride, and stainless steel.

4. The coating method as claimed in claim 1, wherein the biosurfactant is produced by at least one strain selected from the group consisting of Pseudomonas aeruginosa, Bacillus amyloliquefaciens and Serratia marcescens.

5. The coating method as claimed in claim 1, wherein the step of oxidising the surface of the material comprises hydroxylating the surface.

6. The coating method as claimed in claim 1, wherein the biosurfactant has at least one carboxylic group and the step of modifying the biosurfactant to promote its reactivity with the silane linker comprises functionalising the carboxylic group of the biosurfactant by generating activated ester in the presence of N-Hydroxysuccinimide under an anhydrous Steglich esterification reaction.

7. The coating method as claimed in claim 1, wherein the biosurfactant has at least one hydroxyl group and the step of modifying the biosurfactant to promote its reactivity with the silane linker comprises functionalising the hydroxyl group of the biosurfactant by replacing it with a chlorine group.

8. The coating method as claimed in claim 7, wherein the step of modifying the biosurfactant to promote its reactivity with the silane linker includes treating the biosurfactant with thionyl chloride and pyridine.

9. The coating method as claimed in claim 1, wherein the silane linker comprises 3-triethoxysilylpropan-1-amine (APTES).

10. The coating method as claimed in claim 1, wherein the biosurfactant comprises at least one compound selected from the group consisting of lipopeptides, glycolipids and glucosamine derivatives.

11. The coating method as claimed in claim 1, wherein the biosurfactant has biofilm-inhibiting activity against at least one strain selected from the group consisting of Escherichia coli, Listeria monocytogenes, Cryptococcus neoformans, Pseudomonas aeruginosa, and Enterococcus faecalis.

12. The coating method as claimed in claim 4, which includes
performing an extraction step to harvest a crude extract of biosurfactant compounds produced by bacterial cells of the strain, the extraction step comprising: growing the bacterial cells of the strain in a culture medium; removing a bulk of the bacterial cells from the culture medium, thereby to yield a supernatant substantially free of the bacterial cells; acidifying the supernatant, thereby to yield the crude extract of the biosurfactant compounds as a precipitate; freeze drying the precipitate; and at least partially purifying the freeze-dried precipitate by solvent extraction, thereby to yield a purified crude extract of the biosurfactant compounds;
recovering a mixture of the biosurfactant compounds from the purified crude extract of biosurfactant compounds by a liquid membrane process; and
fractionating the mixture of the biosurfactant compounds to obtain fractions thereof, each fraction containing a different constituent biosurfactant compound of the mixture.

13. A method of inhibiting formation of a biofilm on the surface of a substrate material comprising utilizing the coating method of claim 1.

14. An article of manufacture comprising a biosurfactant covalently bonded to a substrate material, wherein the substrate material is selected from the group consisting of polymers and ferrous metals; and the biosurfactant comprises at least one compound selected from the group consisting of lipopeptides, glycolipids and glucosamine derivatives.

15. An article of manufacture comprising a substrate material at least partially coated with a biosurfactant, wherein the substrate material is selected from the group consisting of high-density polyethylene, polyvinyl chloride, and stainless steel; and the biosurfactant is produced by at least one strain selected from the group consisting of Pseudomonas aeruginosa, Bacillus amyloliquefaciens and Serratia marcescens.
